(19) United States
(12) Patent Application Publication
(10) Pub. No.: US 2024/0264159 A1
(43) Pub. Date: Aug. 8, 2024

(54) SYSTEM, METHOD AND KITS FOR THE DETECTION OF BINDING AGENTS

(71) Applicant: AB VALIDATION INC. FAS IVANO BIOSCIENCE, Québec (CA)

(72) Inventors: Mathias MANGION, Québec (CA); Juliette CHAMPEIL, Québec (CA)

(21) Appl. No.: 18/564,895

(22) PCT Filed: Dec. 23, 2022
(86) PCT No.: PCT/CA2022/051896

§ 371 Date: Nov. 28, 2023

Related U.S. Application Data
(60) Provisional application No. 63/265,944, filed on Dec. 23, 2021.

(51) Int. Cl. G01N 33/569; G01N 21/64; G01N 33/543

(52) U.S. Cl. CPC G01N 33/56983; G01N 21/6428; G01N 21/6458; G01N 33/5432; G01N 33/54386; G01N 33/56911; G01N 2021/6439; G01N 2333/165; G01N 2333/32; G01N 2469/20

(57) ABSTRACT

Described herein are cell-free in vitro systems, methods, and kits relating to the detection of binding agents in a fluid sample that disrupt host-microbe interaction, fusion, and/or penetration. The systems, methods, and kits described herein include host nanoparticles expressing a surface receptor recognized by a foreign ligand, and foreign nanoparticles expressing the foreign ligand, wherein preincubation of either the host or foreign nanoparticles with the fluid sample decreases binding/interaction between the host nanoparticles and the foreign nanoparticles when binding agents are present in the fluid sample.

The present description relates to cell-free and pathogen-free systems, methods, and kits for the detection of binding agents. The present description further relates to the use of host and foreign nanoparticles expressing surface receptors and ligands mimicking host-pathogen interactions for detecting binding agents present in a biological sample.

### BACKGROUND

The detection of neutralizing agents (e.g., neutralizing antibodies) is a critical component in research and development applications for infectious diseases for both human and veterinary medicine, particularly in vaccine and therapeutics development, epidemiological surveillance, herd immunity surveillance, contact tracing (duration of immunity), and monitoring of marketed vaccines against new variants. Particularly in the context of rapidly evolving infectious diseases such as the SARS-CoV-2 pandemic and emergence of new variants, methods for detecting neutralizing agents in human samples is important for controlling the spread of disease. Existing methods and kits for detecting neutralizing agents, particularly for the detection of pathogen neutralization agents or blocking molecules, do not closely mimic the physiological conditions of host-pathogen interactions in vitro. Furthermore, these methods are often time-consuming and rely on the production of recombinant proteins and monoclonal and/or polyclonal antibodies, which may reduce the sensitivity or specificity of the assay. Thus, improved methods for detecting neutralizing agents that are rapid, fully customizable and that closely replicate host-pathogen interactions while being safe to use outside of a biosafety level 2 environment would be highly desirable.

### SUMMARY

In a first aspect, described herein is an in vitro system for the detection of binding agents (e.g., neutralizing or blocking agents that disrupt host-microbe interaction and/or fusion/penetration) in a fluid sample (e.g., biological fluid sample). The system generally comprises: host nanoparticles comprising fragments of host cells expressing or engineered to express surface receptors recognized by a foreign ligand; and foreign nanoparticles expressing or engineered to express the foreign ligand on their surface such that the foreign ligands are recognizable by the surface receptors of the host nanoparticles; wherein the host nanoparticles and/or the foreign nanoparticles further comprise a detectable label, and wherein said binding agents are present in the sample when preincubation of either the host or foreign nanoparticles with the sample decreases binding/interaction between the host nanoparticles and the foreign nanoparticles as compared to a corresponding fluid sample lacking the binding agents.

In a further aspect, described herein is a kit for the screening a sample for the presence of binding agents (e.g., that disrupt host-microbe interaction and/or fusion/penetration). The kit generally comprises: a first container comprising host nanoparticles comprising or consisting of fragments of mammalian host cells expressing a surface receptor recognized by a foreign ligand; and a second container comprising foreign nanoparticles expressing the foreign ligands on their surface such that the foreign ligands are recognizable by the surface receptors of the host nanoparticles, wherein the host nanoparticles and/or the foreign nanoparticles further comprise a detectable label, and wherein said binding agents are present in the sample when preincubation of either the host or foreign nanoparticles with the sample decreases binding/interaction between the host nanoparticles and the foreign nanoparticles as compared to a corresponding sample lacking the binding agents.

In a further aspect, described herein is an in vitro method for detecting binding agents (e.g., neutralizing or blocking agents that disrupt host-microbe interaction and/or fusion/penetration) in a fluid sample (e.g., biological fluid sample). The method generally comprises: providing the host nanoparticles and foreign nanoparticles as defined herein; preincubating the host nanoparticles or the foreign nanoparticles with the fluid sample to enable binding/interaction of the binding agents; incubating the host nanoparticles with the foreign nanoparticles and measuring binding/interaction therebetween, wherein a decrease in the level of binding/interaction between the host nanoparticles and the foreign nanoparticles following the preincubation with the fluid sample, as compared to preincubation with a corresponding fluid sample lacking the binding agents, is indicative of the presence of binding agents in the fluid sample.

The present description refers to a number of documents, the contents of which is herein incorporated by reference in their entirety.

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one,” but it is also consistent with the meaning of “one or more”, “at least one”, and “one or more than one”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “about” is used to indicate that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

Other objects, advantages and features of the present description will become more apparent upon reading of the following non-restrictive description of specific embodiments thereof, given by way of example only with reference to the accompanying drawings.

### BRIEF DESCRIPTION OF THE DRAWINGS

In the appended drawings:

FIGS. 1A and 1B shows a schematic model of a binding agent detection assay including a host nanoparticle (H) coated on the well of a microplate expressing surface host receptor, a fluorescent foreign nanoparticle (F) expressing a surface ligand specific for the host receptor, and a neutralizing agent, according to one example embodiment. FIG. 1A shows a neutralizing agent (e.g., an antibody) being specific to the foreign ligand of the foreign nanoparticles, thereby preventing recognition to a corresponding surface receptor on the host nanoparticle. FIG. 1B shows a blocking agent being specific to the surface receptor of the host nanoparticles (i.e., blocking), thereby preventing recognition to a corresponding foreign ligand on the foreign nanoparticle.

FIG. 2A shows schematic model of a vesicle form of the host nanoparticle H having a variety of proteins expressed on the plasma membrane, as well as intracellular-expressing proteins. FIG. 2B shows a schematic of a membrane form of the host nanoparticle H having a variety of proteins expressed on the plasma membrane.

FIG. 3A shows schematic model of a vesicle form of the foreign nanoparticle F having a variety of proteins expressed on the plasma membrane, as well as intracellular-expressing proteins. FIG. 3B shows a schematic of a membrane form of the foreign nanoparticle F having a variety of proteins expressed on the plasma membrane. FIG. 3C shows schematic model of a vesicle form of the foreign nanoparticle F being a capsid, and having a variety of proteins expressed on the plasma membrane, as well as intracellular-expressing proteins

FIG. 4A-4F shows schematic models of different types of host-foreign nanoparticle interactions in the assay. FIG. 4A shows the binding of vesicle forms of host and foreign nanoparticles. FIG. 4B shows the binding of vesicle forms of host and foreign nanoparticles, and subsequent fusion of the membranes and transfer of intracellular content and signal proteins (e.g., transfer of foreign intracellular signal proteins to the host). FIG. 4C shows the binding of vesicle forms of host and foreign nanoparticles, subsequent fusion of the membranes, transfer of intracellular content and proteins, and binding of host and foreign intracellular proteins to form a functioning signal. FIG. 4D shows the binding of membrane forms of host and foreign nanoparticles. FIG. 4E shows the binding of the membrane form of the foreign nanoparticles to the vesicle form of the host nanoparticle. FIG. 4F shows the binding of the vesicle form of the foreign nanoparticles to the membrane form of the host nanoparticle.

FIG. 5A shows a representative fluorescent microscopy of HEK293T cells incubated with control fluorescent viral nanoparticles lacking a ligand for binding HEK293T cells. No binding was observed with increasing amounts of viral nanoparticle. FIG. 5B shows a representative fluorescent microscopy of binding of vesicular stomatitis virus (VSV) model of fluorescent nanoparticles with increasing amounts of viral nanoparticle to HEK293T cells. FIG. 5C shows the quantification of binding by flow cytometry of FIGS. 5A and 5B with increasing amounts of viral nanoparticle.

FIG. 6A shows a representative fluorescent microscopy of HEK293T cells incubated with control fluorescent VSV viral nanoparticles expressing a plasmid containing a trans-activatable GFP-expression cassette. No binding and therefore no internalization (i.e., no fluorescence) was observed with increasing amounts of viral nanoparticle. FIG. 6B shows a representative fluorescent microscopy of binding and internalization of vesicular stomatitis virus (VSV) model of fluorescent nanoparticles expressing a plasmid containing a trans-activatable GFP-expression cassette with increasing amounts of viral nanoparticle into HEK293T cells. FIG. 6C shows the quantification of binding by flow cytometry of FIGS. 6A and 6B with increasing amounts of viral nanoparticle.

FIG. 7 shows a representative flow cytometry analysis of HEK293T cells incubated with a mix of the vesicular stomatitis virus (VSV) model of fluorescent nanoparticles with different concentrations of an anti-VSV neutralizing antibody (Ab01402-2.0).

FIG. 8 shows a representative flow cytometry analysis of HEK293T cells incubated with a mix of VSV-G-pseudotyped lentiviruses encapsulating a trans-activatable GFP-expression cassette with different concentrations of an anti-VSV neutralizing antibody (Ab01402-2.0).

FIG. 9 shows a representative flow cytometry analysis of binding of VSV-like viral nanoparticles to host nanoparticles expressing surface receptors specific to VSV glycoproteins.

FIG. 10A-10D shows a representative flow cytometry analysis of host nanoparticles expressing angiotensin converting enzyme 2 (ACE2) receptors incubated with a mix of a SARS-CoV-2 model of fluorescent viral nanoparticles with different concentrations of either an irrelevant antibody (anti-VSV antibody; Ab01402-2.0) or a neutralizing anti-Spike RBD (receptor binding domain) antibody (Ab02019-12.1) (FIG. 10A), either FBS or pooled anti-SARS-CoV-2 antibody positive human sera (FIG. 10B), purified neutralizing chicken IgY polyclonal antibody against the SARS-CoV-2 spike protein S1 region (FIG. 10C), or purified neutralizing chicken IgY polyclonal antibody against the spike protein S2 region of SARS-CoV-2 (FIG. 10D).

FIG. 11A shows the results of a fusion assay between R18-labeled SARS-CoV-2 foreign enveloped nanoparticles and host ACE2 nanoparticles, in suspension, in either PBS or DMEM. FIG. 11B shows the results of a fusion assay between R18-labeled SARS-CoV-2 foreign enveloped nanoparticles and host ACE2/TMPRSS2 nanoparticles, in suspension, in either PBS or DMEM. The fluorescent signal is expressed in relative fluorescent unit (RFU) normalized to controls.

FIG. 12 shows the results of a fusion assay between R18-labeled SARS-CoV-2 foreign enveloped nanoparticles and host ACE2 nanoparticles or host ACE2/TMPRSS2 nanoparticles, in suspension, at either 37° C. or 4° C. The fluorescent signal is expressed in relative fluorescent unit (RFU) normalized to controls.

FIG. 13 shows the results of a neutralization assay of the fusion between R18-labeled SARS-CoV-2 foreign enveloped nanoparticles and host ACE2 nanoparticles, in suspension, when untreated or in the presence of either MSA5 aptamer (10 μM) or anti-Spike RBD antibody (20 μg/ml). The fluorescent signal is expressed in relative fluorescent unit (RFU) normalized to controls.

FIG. 14 shows the results of a fusion assay between SARS-CoV-2 foreign enveloped nanoparticles and coated host ACE2 nanoparticles, and subsequent neutralization in the presence of either anti-Spike RBD antibody (10 μg/ml) or anti-VSVg antibody (10 μg/ml).

FIG. 15A shows the results of a neutralization assay between SARS-CoV-2 foreign enveloped nanoparticles and coated host ACE2 nanoparticles in the presence of either anti-Spike RBD antibody (20 μg/ml), soluble ACE2 receptor (10 μg/ml), Ouabain (120 nM), MSA5 aptamer (10 μM), soluble Spike RBD (10 μg/ml), or anti-VSVg antibody (10 μg/ml). FIG. 15B shows the results of a neutralization assay between coated SARS-CoV-2 foreign enveloped nanoparticles and host ACE2 nanoparticles in the presence of either anti-Spike RBD antibody (20 μg/ml), soluble ACE2 receptor (10 μg/ml), or soluble Spike RBD (10 μg/ml). Results are expressed in neutralization % of relative fluorescent unit (RFU) normalized to controls.

FIG. 16 shows the results of a neutralization assay between SARS-CoV-2 foreign enveloped nanoparticles and coated host ACE2 nanoparticles in the presence of either anti-Spike RBD (10 μg/ml) or anti-VSVg antibody (10 μg/ml) diluted in either a solution containing urine (1/50), saliva (1/20), serum (1/20), or plasma (1/50). Results are expressed in neutralization % of relative fluorescent unit (RFU) normalized to controls.

FIG. 17A shows the results of a neutralization assay between SARS-CoV-2 foreign enveloped nanoparticles and coated host ACE2 nanoparticles in the presence of either anti-Spike RBD (10 μg/ml) or anti-VSVg antibody (10 μg/ml), whereby the assay was conducted at 37° C. or room temperature using the incubation times as previously described (1 hour pre-incubation of antibody and foreign nanoparticles; 3 hour incubation of host and foreign nanoparticles/antibody mix). FIG. 17B shows the results of a neutralization assay between SARS-CoV-2 foreign enveloped nanoparticles and coated host ACE2 nanoparticles in the presence of either anti-Spike RBD (10 μg/ml) or anti-VSVg antibody (10 μg/ml), whereby the assay was conducted at 37° C. or room temperature and the SARS-CoV-2 nanoparticles and antibody pre-incubation period was reduced to 30 minutes from 1 hour. FIG. 17C shows the results of a neutralization assay between SARS-CoV-2 foreign enveloped nanoparticles and coated host ACE2 nanoparticles in the presence of either anti-Spike RBD (10 μg/ml) or anti-VSVg antibody (10 μg/ml) whereby the assay was conducted at 37° C. or room temperature and the host ACE2 nanoparticles SARS-CoV-2 nanoparticles/antibody incubation period was reduced to 1 hour from 3 hours. FIG. 17D shows the results of a neutralization assay between SARS-CoV-2 foreign enveloped nanoparticles and coated host ACE2 nanoparticles in the presence of either anti-Spike RBD (10 μg/ml) or anti-VSVg antibody (10 μg/ml) whereby the assay was conducted at 37° C. or room temperature, the SARS-CoV-2 nanoparticles and antibody pre-incubation period was reduced to 30 minutes from 1 hour, and the host ACE2 nanoparticles SARS-CoV-2 nanoparticles/antibody incubation period was reduced to 1 hour from 3 hours. Results are expressed in neutralization % of relative fluorescent unit (RFU) normalized to controls.

FIG. 18 shows the results of a neutralization assay between Bacillus anthracis foreign nanoparticles and coated host ANTXR2 nanoparticles in the presence of either anti-PA63 antibody (10 μg/ml) or anti-VSVg antibody (10 μg/ml). Results are expressed in neutralization % of relative fluorescent unit (RFU) normalized to controls.

FIG. 19A shows the results of a neutralization assay between foreign non-enveloped norovirus nanoparticles expressing surface VP1 protein and coated host FUT2 nanoparticles in the presence of either anti-VP1 antibodies (10 μg/ml or 100 μg/ml) or anti-VSVg antibody (10 μg/ml) (FIG. 19B). Results are expressed in neutralization % of relative fluorescent unit (RFU) normalized to controls.

FIG. 20 shows the results of a neutralization assay between SARS-CoV-2 foreign enveloped nanoparticles and coated host ACE2 nanoparticles produced from HeLa cells, in the presence of either anti-Spike RBD antibody (10 μg/ml or 100 μg/ml). Results are expressed in neutralization % of relative fluorescent unit (RFU) normalized to controls.

FIG. 21 shows representative transmission electron microscopy images of Spike-SARS-CoV-2 foreign enveloped nanoparticles (FIG. 21A), ACE2 host enveloped nanoparticles (FIG. 21B), norovirus VP1 foreign non-enveloped nanoparticles (FIG. 21C), and linearized spike-SARS-CoV-2 foreign enveloped nanoparticles (FIG. 21D).

### DETAILED DESCRIPTION

Described herein are cell-free and/or pathogen-free in vitro systems, methods, and kits relating to the detection of binding agents in a biological sample. In some aspects, the present invention stems from the production of host and foreign nanoparticles that can be used to mimic host-pathogen interactions in vitro and to detect neutralization agents that are capable for blocking the interaction of the host and foreign nanoparticles.

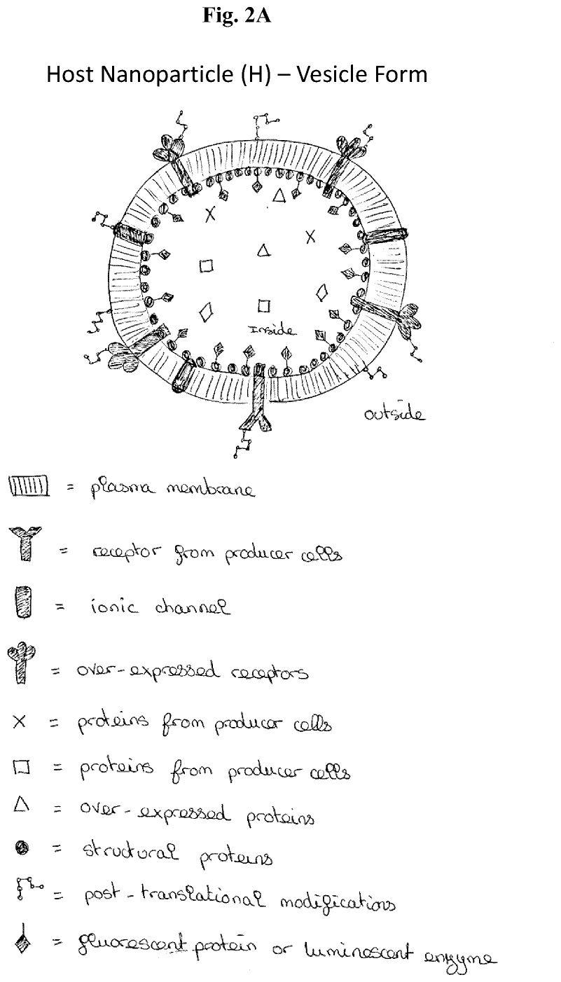

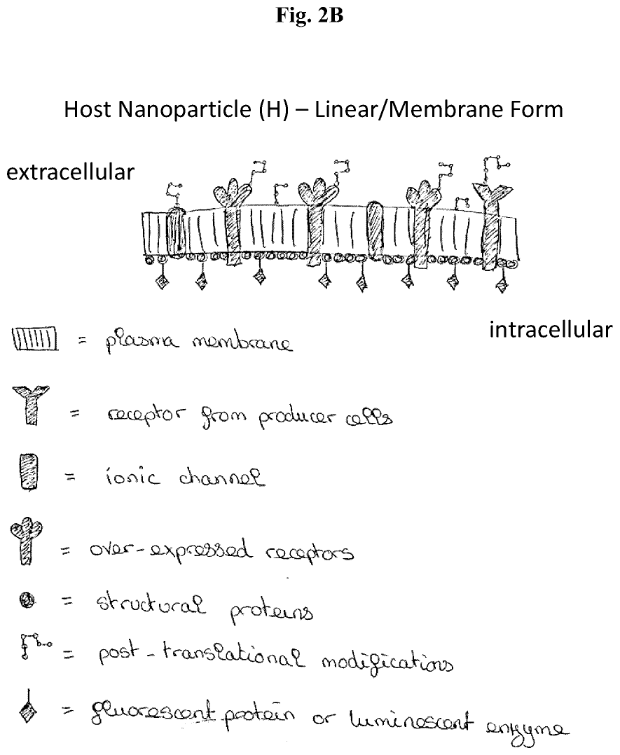

As used herein, the term “host nanoparticles” refers to nanoparticles that are produced or derived from producer or host cells that natively or have been engineered to express or overexpress one or more surface receptors or proteins that recognize a foreign ligand. Host nanoparticles are preferably engineered to be replication-deficient by lacking machinery required for replication (e.g., a nucleus). For example, host nanoparticles may be derived or produced from eukaryotic or mammalian cells (e.g., cell lines or ex vivo cells). The host nanoparticles may include a plasma membrane, and can be in a vesicular (FIG. 2A) having an “intracellular” portion, or can be linear form (FIG. 2B). In some embodiments, the host nanoparticles consist of or include proteins derived from the producer cells. The host nanoparticles may include other surface (e.g., located on the plasma membrane) or intracellular proteins such as ionic channels, structural proteins/fatty acids/carbohydrates, or detectable molecules (e.g., fluorescent or luminescent proteins), or proteins required for producing a functional detectable molecule. In some embodiments, the surface or intracellular proteins expressed include all of the physiological post-translational modifications (e.g., phosphorylation, glycosylation, ubiquitination, nitrosylation, methylation, acetylation, or lipidation). In a SARS-CoV-2 model, for example, the surface receptors/proteins on the host nanoparticle may be angiotensin converting enzyme 2 (ACE2) receptor and/or transmembrane serine protease 2 (TMPRSS2). The host nanoparticles may be of any size ranging between 1 nm to 500 nm.

In some embodiments, the host producer cells are eukaryotic (e.g., yeast or mammalian) cells. In some embodiments, the host producer cells may be engineered to express or overexpress the foreign ligand. In some embodiments, the host producer cells may be engineered to express or overexpress the detectable label. In some embodiments, the host producer cells may be engineered to express or overexpress an exogenous viral (e.g., from an enveloped or non-enveloped virus), bacterial, or fungal protein that induces extracellular vesicle or extracellular particle formation (e.g., biological nanoparticles or virus-like particles) in the host producer cell.

In some embodiments, the host nanoparticles are vesicular nanoparticles comprising an exogenous viral structural protein (e.g., a late assembly (L) domain protein or polyprotein, such as HIV Gag) that induces host cell budding. In some embodiments, the exogenous viral structural protein may be fused to the detectable label.

As used herein, the term “foreign nanoparticles” generally refers to nanoparticles that are designed to mimic a microbe or pathogen that interacts with, fuses and penetrates, or infects a host cell. In some embodiments, foreign nanoparticles natively or are engineered to express or overexpress foreign ligands that are specific to receptors present on the surface of host nanoparticles. Foreign nanoparticles are preferably engineered to be replication-deficient by lacking machinery required for replication. For example, the foreign nanoparticles may be derived or produced from a microorganism, such as but not limited to any pathogen like bacteria, fungi, viruses, or parasites. In some embodiments, the foreign nanoparticles may be produced or derived from eukaryotic (e.g., yeast or mammalian) producer cells. In some embodiments, the foreign nanoparticles may be formed by self-assembling viral structural proteins (e.g., capsid proteins).

In some embodiments, the microorganism or eukaryotic producer cells from which the foreign nanoparticles are produced or derived are may be: engineered to express or overexpress the surface receptor; engineered to express or overexpress the detectable label; engineered to express or overexpress an exogenous viral (e.g., from an enveloped or non-enveloped virus), bacterial, or fungal protein that induces extracellular vesicle or extracellular particle formation (e.g., biological nanoparticles or virus-like particles) in the host producer cell; nanoparticles that fuse with and/or penetrate into the host nanoparticles upon contact therewith; or any combination thereof.

In some embodiments, the foreign nanoparticles may be vesicular foreign nanoparticles comprising an exogenous viral structural protein (e.g., a late assembly (L) domain protein or polyprotein, such as HIV Gag) that induces host cell budding.

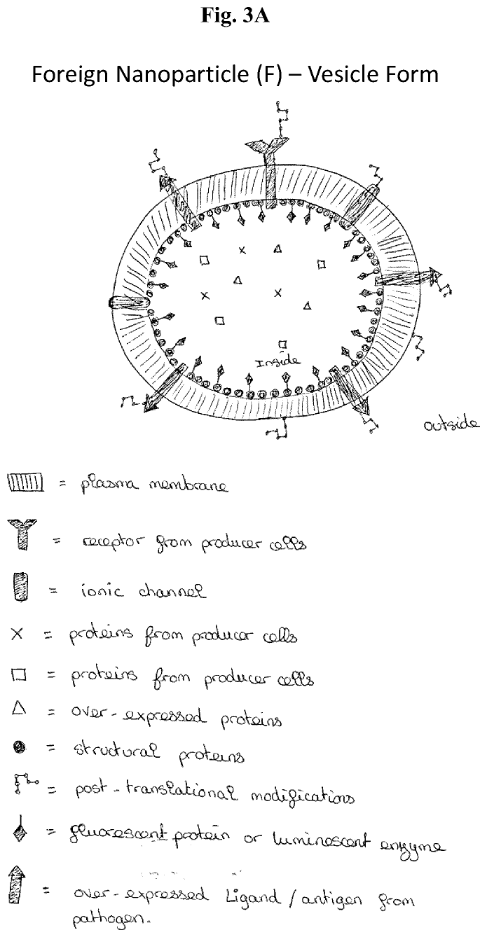

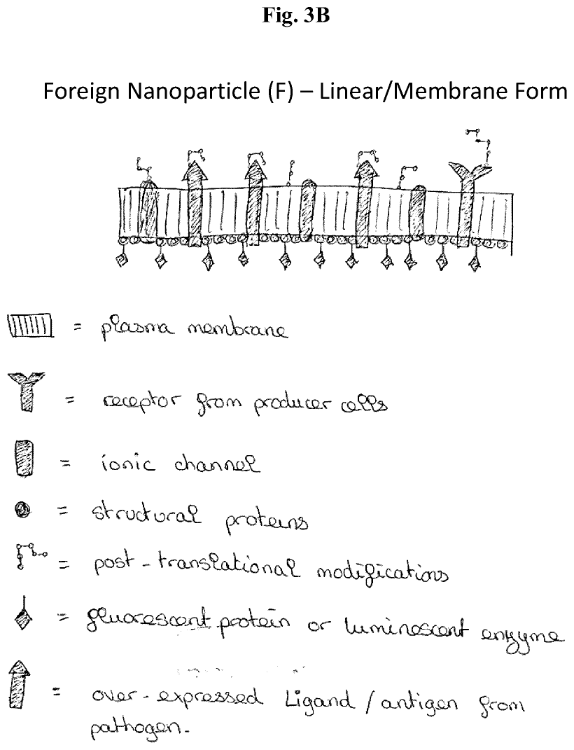

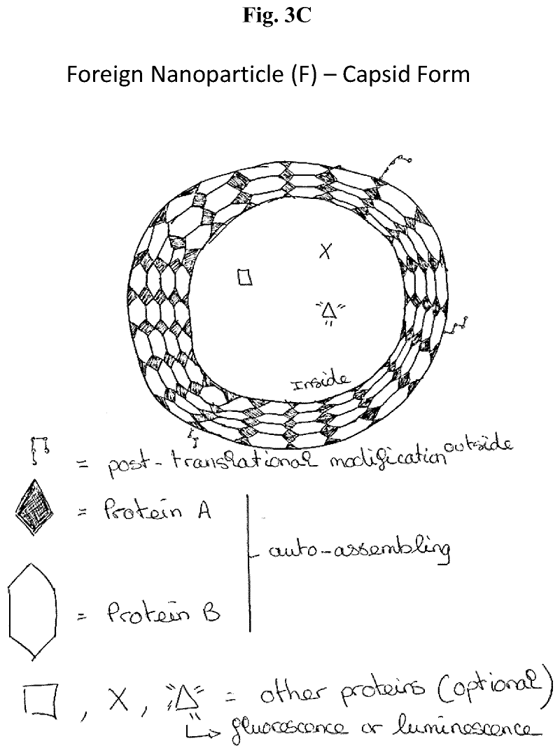

In some embodiments, the foreign nanoparticles are engineered to express or overexpress any surface antigen, autoantigen, or allergen for recognition by a corresponding receptor on the host nanoparticle. The foreign nanoparticles may include a membrane (e.g., plasma membrane), and can be in a vesicular (FIG. 3A) (e.g., capsid form; FIG. 3C) having an “intracellular” portion, or can be linear form (FIG. 3B). The foreign nanoparticles may include other surface (e.g., located on the plasma membrane) or intracellular proteins such as ionic channels, structural proteins/fatty acids/carbohydrates, or detectable molecules (e.g., fluorescent or luminescent proteins), or proteins required for producing a functional detectable molecule. In some aspects, the surface or intracellular proteins expressed include all of the physiological post-translational modifications (e.g., phosphorylation, glycosylation, ubiquitination, nitrosylation, methylation, acetylation, or lipidation). In a SARS-CoV-2 model, for example, the foreign ligand may be the full-length Spike protein, S1, S2, or the receptor binding domain (RBD) of the Spike protein. The foreign nanoparticles may be of any size ranging between 1 nm to 500 nm.

As used herein, the term “detectable molecule” or “detectable label” refers to a molecule that is readily detectable or that can be converted or modified to be detectable. For example, the detectable molecule may any fluorescent (e.g., GFP) or luminescent (e.g., chemiluminescent) molecule (i.e., directly detectable). In some aspects, the detectable molecule can initially be precursor molecule that is converted or modified into a functional detectable by any modification (e.g., chemical). For example, the detectable molecule may be a gene encoding a fluorescent molecule that is translated into a functional fluorescent protein (e.g., GFP, luciferase, secretory alkaline phosphatase [SEAP]). In some aspects, conversion or modification of the detectable molecule by proteins or machinery present or expressed in the host nanoparticle (e.g., either on the surface or intracellular) occurs upon binding of the foreign nanoparticle to the host nanoparticle and/or subsequent internalization of the foreign nanoparticle. In some embodiments, the detectable label is detectable upon contact with a substrate, protein, or enzyme (i.e. indirectly detectable).

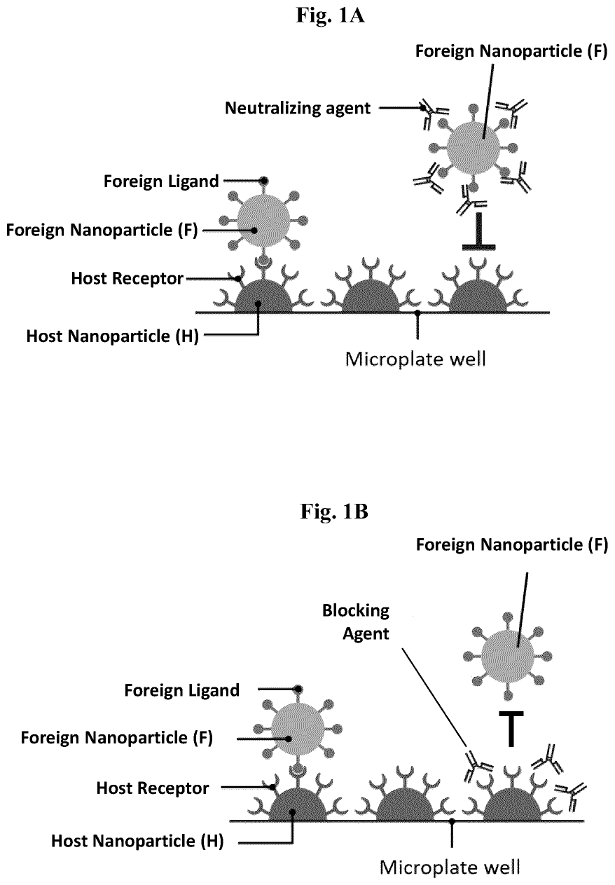

As used herein, the term “binding agent” refers to a molecule that is capable of preventing recognition of a foreign ligand of a foreign nanoparticle to its corresponding surface receptor on host nanoparticles (FIG. 1A) (e.g., neutralizing agent). In some aspects, the binding agent is a blocking agent. In some aspects, the binding agent is specific to the surface receptor of the host nanoparticle or to the foreign ligand of the foreign nanoparticle (FIG. 1B) (e.g., blocking agent). In some aspects, the binding agent is an antibody or any antigen binding fragment thereof. In some aspects, the antibody is any monoclonal, polyclonal, chimeric, natural, unnatural, recombinant, isotype, or species of antibody. In some aspects, the neutralizing antibody is a natural or synthetic peptide, protein, nucleic acid (e.g., aptamer or ribozyme), or chemical molecule (e.g., small molecule). In some embodiments, the binding agent may inhibit the fusion of host and foreign nanoparticle membranes. In some aspects, the binding agent may inhibit the internalization or penetration of the foreign nanoparticle into the host nanoparticle.

As used herein, the term “sample” refers to any sample, particularly a fluid sample, suspected to include a binding agent. The sample may be a any laboratory or biological. Samples can include but are not limited to cellular extracts, extracellular fluid, fluid harvested from the body of a subject (e.g., animal or human), culture media, blood, bone marrow, plasma, serum, tears, feces, saliva, nasal secretion, bronchoalveolar lavage fluid (BALF), spinal fluid, biopsy, or any organ. In some aspects, the sample may be any solution containing one or more candidate binding agents to be screened by the methods, systems, or kits defined herein.

In some aspects, the in vitro systems described herein for the detection of binding agents (e.g., neutralizing or blocking agents that disrupt host-microbe interaction) in a fluid sample (e.g., biological fluid sample), generally include host nanoparticles comprising plasma membrane fragments of host cell expressing or engineered to express surface receptors recognized by a foreign ligand; foreign nanoparticles expressing or engineered to express the foreign ligand on their surface such that the foreign ligands are recognizable by the surface receptors of the host nanoparticles; wherein the host nanoparticles and/or the foreign nanoparticles further comprise a detectable label, and wherein said binding agents are present in the sample when preincubation of either the host or foreign nanoparticles with the sample decreases binding/interaction between the host nanoparticles and the foreign nanoparticles as compared to a corresponding fluid sample lacking the binding agents. FIGS. 1A and 1B show example embodiments of the system described herein.

In further aspect, the kits described herein for the screening a sample for the presence of binding agents (e.g., that disrupt host-microbe interaction), generally include a first container comprising host nanoparticles comprising or consisting of plasma membrane fragments of mammalian host cells expressing a surface receptor recognized by a foreign ligand; and a second container comprising foreign nanoparticles expressing the foreign ligands on their surface such that the foreign ligands are recognizable by the surface receptors of the host nanoparticles, wherein the host nanoparticles and/or the foreign nanoparticles further comprise a detectable label, and wherein said binding agents are present in the sample when preincubation of either the host or foreign nanoparticles with the sample decreases binding/interaction between the host nanoparticles and the foreign nanoparticles as compared to a corresponding sample lacking the binding agents.

In a further aspect, the in vitro methods described herein for detecting binding agents (e.g., neutralizing or blocking agents that disrupt host-microbe interaction) in a fluid sample (e.g., biological fluid sample), generally include providing the host nanoparticles and foreign nanoparticles as defined herein; preincubating the host nanoparticles or the foreign nanoparticles with the fluid sample to enable binding/interaction of the binding agents; incubating the host nanoparticles with the foreign nanoparticles and measuring binding/interaction therebetween, wherein a decrease in the level of binding/interaction between the host nanoparticles and the foreign nanoparticles following the preincubation with the fluid sample, as compared to preincubation with a corresponding fluid sample lacking the binding agents, is indicative of the presence of binding agents in the fluid sample.

In some aspects, the binding/interaction between the host nanoparticles and the foreign nanoparticles refers to the ability of binding agents to neutralize foreign nanoparticles (neutralization activity), block host nanoparticles receptors (blocking activity), inhibit fusion of the host and foreign nanoparticle membranes, and/or inhibit the internalization/penetration of the foreign nanoparticles into the host nanoparticles. In some aspects, the systems, methods, or kits described herein include a blocking step using any appropriate blocking solution for reducing non-specific binding (e.g., bovine serum albumin, milk, or FBS). In some aspects, systems and methods described herein include one or more washing steps, particularly after incubating the foreign nanoparticles with the host nanoparticles to remove unbound/uninternalized (e.g., neutralized via the binding agent) foreign nanoparticles. In some aspects, the detection of detectable molecule may be done by any common method/instrument for detecting luminescence or fluorescence.

In some aspects, the host nanoparticles and/or the foreign nanoparticles may immobilized or are for immobilization on a solid support. In some aspects, the solid support may be any suitable surface for coating or hybridizing such as a microplate, tube, or on beads. The host nanoparticles may be precoated on the support or coated by known methods for coating that are used in immunoassays, such as in ELISAs. In some aspects, addition of the foreign nanoparticles to the host nanoparticles may be done in the presence of the binding agent (e.g., a preincubation step) or may be done after incubation of the host nanoparticles with the binding agent (e.g., a blocking step). The steps of the methods and systems described herein can be done generally between 4° C. and 37° C. (e.g., room temperature) in the presence or absence of CO₂ (e.g., 5%). For example, the coating step may be done at any temperature between 4° C. and 37° C. for a period of time ranging between 1 to 48 hours. Incubation of host and foreign nanoparticles (or preincubated with binding agent) may be conducted at any temperature between 4° C. and 37° C. for a period of time ranging between 0.5 to 8 hours.

In some aspects, the host nanoparticles and/or the foreign nanoparticles may be in suspension.

In some aspects, the host nanoparticles and/or the foreign nanoparticles, and subsequent incubations for detecting the binding agents, may be in any solution or gels (e.g., hydrogels), such as aqueous solutions, buffers (e.g., PBS), or medium (e.g., DMEM).

In some aspects, the absence or decrease in the detection of the detectable molecule is indicative of the presence of a binding agent. In some aspects, the amount or concentration of the binding agent negatively correlates with the amount of detectable molecule detected. For example, increasing concentrations of binding agent may bind to the host or foreign nanoparticle and prevent binding of the foreign nanoparticle having a detectable molecule. Upon a subsequent washing a step, the unbound/uninternalized foreign nanoparticles are washed out of the system, thereby decreasing the detection of the detectable molecule. In some aspects, the binding agent present in a sample can be determined quantitatively (e.g., by using a standard curve), semi-quantitatively, or qualitatively (e.g., presence or absence).

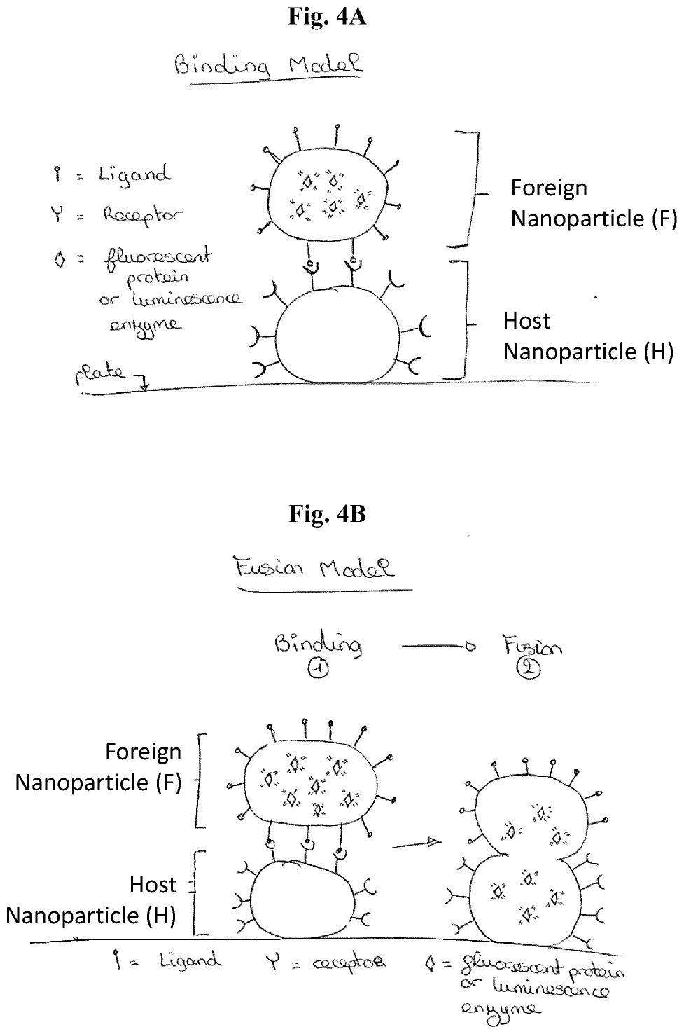

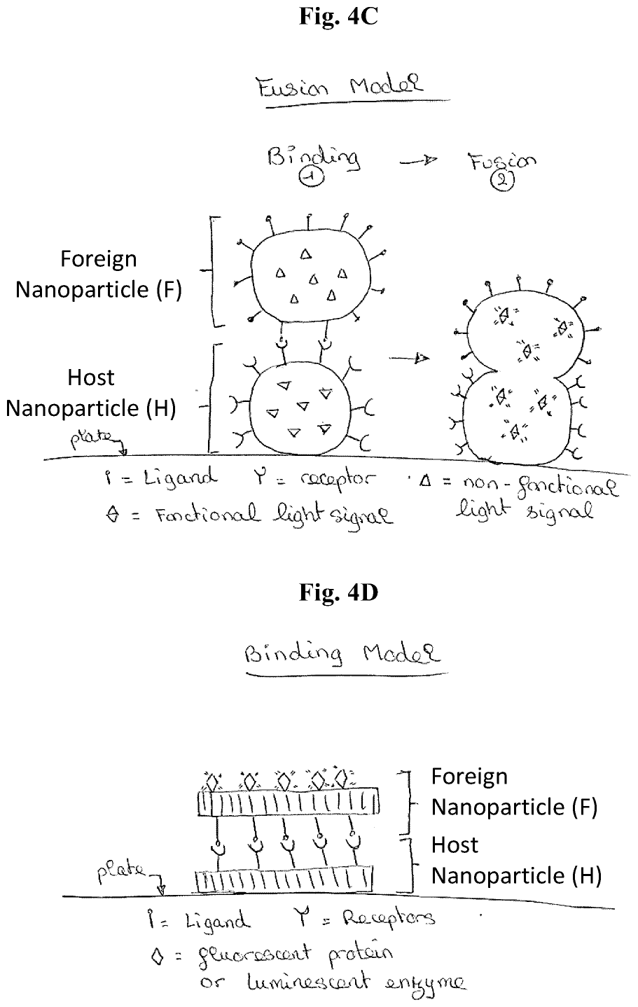

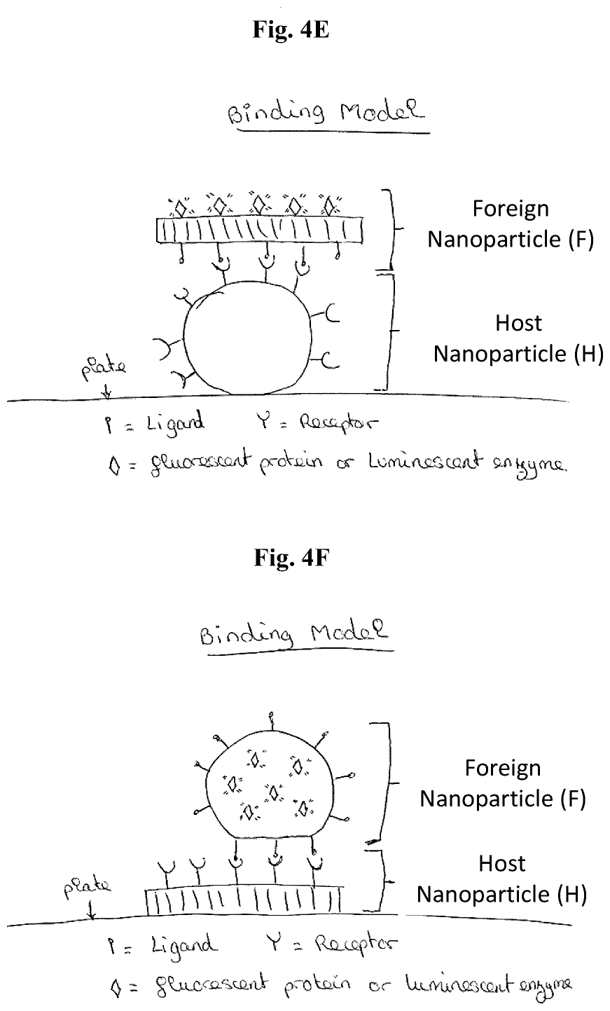

In some aspects, the foreign nanoparticles bind the host nanoparticles, either vesicular or membrane forms, via foreign ligand and surface receptor recognition (FIG. 4A, 4D, 4E, or 4F). In some cases, binding of the foreign nanoparticles to the host nanoparticles induces fusion of the two respective membranes (FIG. 4B or 4C). In some cases, fusion of the membranes is followed by one or two-way transfer of intracellular contents (e.g., proteins). In some cases, binding of the foreign nanoparticles to the host nanoparticles induces internalization of the foreign nanoparticles. In some cases, internalization is followed by destruction of the foreign nanoparticle membrane and release of its intracellular contents. For example, the detectable molecule may be transferred to the host nanoparticle or may be converted to a functional detectable molecule upon transfer.

The principle of the methods and systems described herein provide several advantages over existing methods and assays for detecting binding agents. First, the systems and methods described herein do not utilize live cells or pathogens (e.g., bacteria or viruses) and therefore are safe for use outside of biosafety level 2 environments. Second, physiological host-pathogen interaction is closely mimicked which promote neutralizing/blocking agent activity, including preserving the post-translational modifications of surface receptors/proteins and foreign ligands (e.g., glycoproteins). Third, the systems and methods described herein provide a rapid response for the presence of binding agent (e.g., 0.5-8 hours). Fourth, the systems and methods described herein may be partially or fully automated.

In some aspects, the kit may include a microplate (e.g., 96-well plate) or tube that is precoated with the host nanoparticle. The kit may further include coating, blocking, washing, diluting, and/or detecting solutions. The host and/or foreign nanoparticles may be included in solution (ready-to-use) or dehydrated/lyophilized (to be reconstituted). The kit may further include one or more positive controls (e.g., a commercial or purified neutralizing antibody) and/or a standard curve, that is in solution or lyophilized. The kit may further include bottle for ready-to-use or reconstituted wash solution. The kit may also include instructions for use.

### Items

In various embodiments, described herein are one or more of the following items:
1. An in vitro system for the detection of binding agents (e.g., neutralizing or blocking agents that disrupt host-microbe interaction and/or fusion/penetration) in a fluid sample (e.g., biological fluid sample), said system comprising: host nanoparticles comprising fragments of host cells expressing or engineered to express surface receptors recognized by a foreign ligand; and foreign nanoparticles expressing or engineered to express the foreign ligand on their surface such that the foreign ligands are recognizable by the surface receptors of the host nanoparticles; wherein the host nanoparticles and/or the foreign nanoparticles further comprise a detectable label, and wherein said binding agents are present in the sample when preincubation of either the host or foreign nanoparticles with the sample decreases binding/interaction between the host nanoparticles and the foreign nanoparticles as compared to a corresponding fluid sample lacking the binding agents.
2. A kit for the screening a sample for the presence of binding agents (e.g., that disrupt host-microbe interaction and/or fusion/penetration), said kit comprising: a first container comprising host nanoparticles comprising or consisting of fragments of mammalian host cells expressing a surface receptor recognized by a foreign ligand; and a second container comprising foreign nanoparticles expressing the foreign ligands on their surface such that the foreign ligands are recognizable by the surface receptors of the host nanoparticles, wherein the host nanoparticles and/or the foreign nanoparticles further comprise a detectable label, and wherein said binding agents are present in the sample when preincubation of either the host or foreign nanoparticles with the sample decreases binding/interaction between the host nanoparticles and the foreign nanoparticles as compared to a corresponding sample lacking the binding agents.
3. An in vitro method for detecting binding agents (e.g., neutralizing or blocking agents that disrupt host-microbe interaction and/or fusion/penetration) in a fluid sample (e.g., biological fluid sample), said method comprising: providing the host nanoparticles and foreign nanoparticles as defined in item 1 or 2; preincubating the host nanoparticles or the foreign nanoparticles with the fluid sample to enable binding/interaction of the binding agents; incubating the host nanoparticles with the foreign nanoparticles and measuring binding/interaction therebetween, wherein a decrease in the level of binding/interaction between the host nanoparticles and the foreign nanoparticles following the preincubation with the fluid sample, as compared to preincubation with a corresponding fluid sample lacking the binding agents, is indicative of the presence of binding agents in the fluid sample.
4. The system, kit, or method of any one of items 1 to 3, wherein the host and/or foreign nanoparticles are vesicular or linear shaped.
5. The system, kit, or method of any one of items 1 to 4, wherein the host nanoparticles are produced or derived from host producer cells.
6. The system, kit, or method of item 5, wherein the host producer cells are eukaryotic (e.g., yeast or mammalian) cells.
7. The system, kit, or method of item 5 or 6, wherein the host producer cells are: (a) engineered to express or overexpress the foreign ligand; (b) engineered to express or overexpress the detectable label; (c) engineered to express or overexpress an exogenous viral (e.g., from an enveloped or non-enveloped virus), bacterial, or fungal protein that induces extracellular vesicle or extracellular particle formation (e.g., biological nanoparticles or virus-like particles) in the host producer cell; or (d) any combination of (a) to (c).
8. The system, kit, or method of any one of items 1 to 7, wherein the host nanoparticles are vesicular nanoparticles comprising an exogenous viral structural protein (e.g., a late assembly (L) domain protein or polyprotein, such as HIV Gag) that induces host cell budding.
9. The system, kit, or method of item 8, wherein the exogenous viral structural protein is fused to the detectable label.
10. The system, kit, or method of any one of items 1 to 9, wherein the foreign nanoparticles are produced or derived from a microorganism (e.g., bacteria, fungi, viruses, or parasites), from eukaryotic (e.g., yeast or mammalian) producer cells, or are formed by self-assembling viral structural proteins (e.g., capsid proteins).
11. The system, kit, or method of item 10, wherein the microorganism or eukaryotic producer cells from which the foreign nanoparticles are produced or derived are: (a) engineered to express or overexpress the surface receptor; (b) engineered to express or overexpress the detectable label; (c) engineered to express or overexpress an exogenous viral (e.g., from an enveloped or non-enveloped virus), bacterial, or fungal protein that induces extracellular vesicle or extracellular particle formation (e.g., biological nanoparticles or virus-like particles) in the host producer cell; (d) nanoparticles that fuse with and/or penetrate into the host nanoparticles upon contact therewith; or (e) any combination of (a) to (d).
12. The system, kit, or method of any one of items 1 to 11, wherein the foreign nanoparticles are vesicular foreign nanoparticles comprising an exogenous viral structural protein (e.g., a late assembly (L) domain protein or polyprotein, such as HIV Gag) that induces host cell budding.
13. The system, kit, or method of any one of item 1 to 12, wherein the foreign nanoparticles are derived from SARS-CoV-2 virus and/or express or are engineered to express a SARS-CoV-2 Spike protein, or a portion thereof, on their surface.
14. The system, kit, or method of any one of items 1 to 13, wherein the host nanoparticles express or are engineered to express angiotensin converting enzyme 2 (ACE2) and/or transmembrane serine protease 2 (TMPRSS2) on their surface.
15. The system, kit, or method of any one of items 1 to 14, wherein the foreign ligand is a surface ligand expressed on a microbial cell, an autoantigen, or an allergen.
16. The system, kit, or method of any one of items 1 to 15, wherein the detectable label is internally expressed or expressed on the surface of the foreign or host nanoparticles.
17. The system, kit, or method of any one of items 1 to 16, wherein the detectable label is a fluorescent or luminescent molecule (e.g., protein).
18. The system, kit, or method of any one of items 1 to 17, wherein the detectable label is detectable upon contact with a substrate, protein, or enzyme.
19. The system, kit, or method of any one of items 1 to 18, wherein the sample is blood, serum, tears, saliva, plasma, urine, nasal secretion, bronchoalveolar lavage fluid (BALF), fecal, or cerebral spinal fluid.
20. The system, kit, or method of any one of items 1 to 19, wherein the binding agent is an antibody or an antigen-binding fragment thereof, peptide, protein, nucleic acid (e.g., aptamers or ribozymes), or a small molecule.
21. The system, kit, or method of any one of items 1 to 20, wherein the binding agent recognizes the surface receptor of the host nanoparticle or the foreign ligand of the foreign nanoparticle.
22. The system, kit, or method of any one of items 1 to 21, wherein the binding agent prevents or attenuates fusion of the membranes of the host nanoparticle and the foreign nanoparticle.
23. The system, kit, or method of any one of items 1 to 22, wherein the binding agent prevents or attenuates internalization/penetration of the foreign nanoparticle into the host nanoparticle.
24. The system, kit, or method of any one of items 1 to 23, wherein the host nanoparticles comprise plasma membrane fragments of the host cell expressing or engineered to express the surface receptors recognized by the foreign ligand.
25. The system, kit, or method of any one of items 1 to 24, wherein the host nanoparticles and/or the foreign nanoparticles are immobilized or are for immobilization on a solid support.
26. The system, kit, or method of any one of items 1 to 24, wherein the host nanoparticles and/or the foreign nanoparticles are in suspension.
27. The kit of any one of items 2 and 4 to 26, further comprising instructions for use.

### EXAMPLES

### Example 1: Production of Host (H) and Foreign (F) Nanoparticles

Two types of host and foreign nanoparticles were produced for this neutralizing and blocking molecules detection assay. They are a complex biological mixed structures that were based on the assembly of biological molecules of various nature to create a membrane composed of lipids, proteins, and/or polysaccharides (glycans). The nanoparticles were made in either: a vesicular form (FIG. 2A, 3A, and 3C) and therefore have an inner compartment and an extracellular surface, or a linear form (FIGS. 2B and 3B). The nanoparticles originated from cultured eukaryotic or prokaryotic producer cells. They can be composed of all the elements conventionally found in a natural plasma membrane such as the different types of lipids, polysaccharides, proteins, as well as natural post-translational modifications associated with those different elements (e.g., phosphorylation, glycosylation, ubiquitination, nitrosylation, methylation, acetylation, lipidation).

To functionalize these complex biological membrane structures, specifically chosen proteins (i.e., antigen, ligand, receptors) were overexpressed in the producer cells (e.g., adherent or cells in suspension) so that they were integrated into those structures, by various techniques such as by expression vectors (e.g., plasmids). In addition, other overexpressed proteins play a part in the manufacturing process of those membrane type complex biological structures. Among those associated proteins, some can generate a fluorescent or light signal. All the proteins composing the complex biological structures, have a three-dimensional conformation and native post-translational modifications. The integration of antigens or ligands into these structures allows the external surface of pathogens to be simulated to form foreign nanoparticles used to design the neutralizing molecules detection assay. Based on the same principle, the integration of receptors and specific proteins into those complex structures allows the external surface of cells to be mimicked to form host nanoparticles used to design the neutralizing molecule detection assays.

Upon expression/over-expression of the protein(s) of interest, the producer cells are cultured for a period of time to induce nanoparticle formation/budding, and the nanoparticles were further harvested by centrifugation, filtration (0.45 μm), and/or chromatography techniques. To concentrate the nanoparticles, ultracentrifugation on sucrose cushion was typically performed. Nanoparticles were then resuspended, quantified in terms of total protein, and preserved at −80° C. Expression of certain proteins, such as HIV Gag protein, along with the protein of interest was found to aid in developing the internal structure of the nanoparticles and triggering of the budding mechanism of nanoparticles from producer cells.

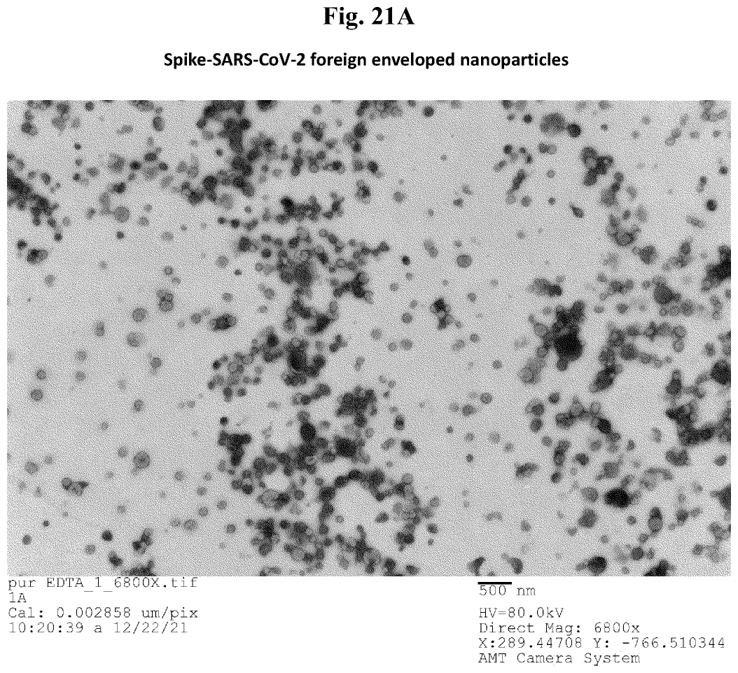

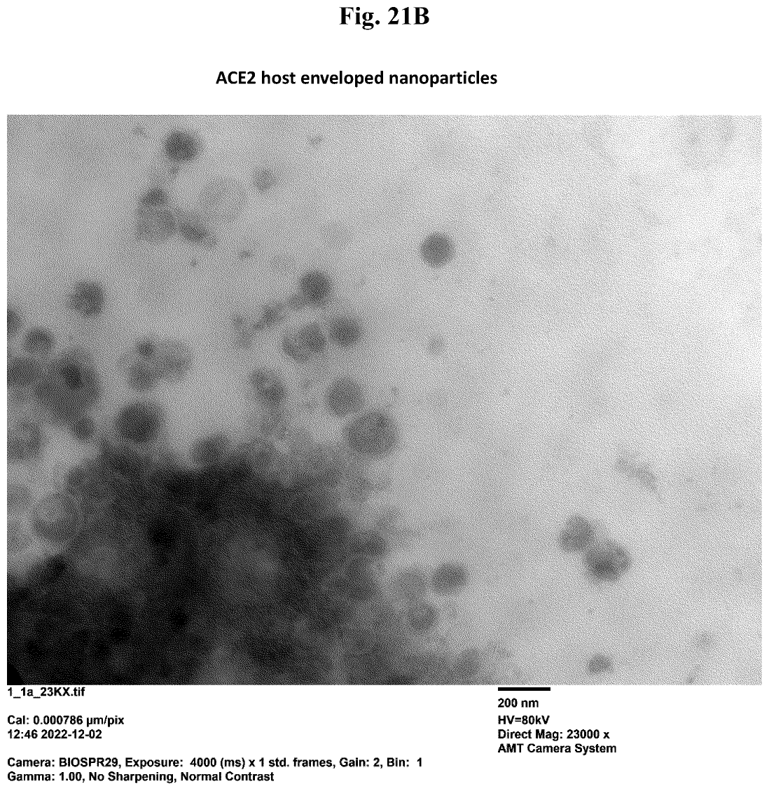

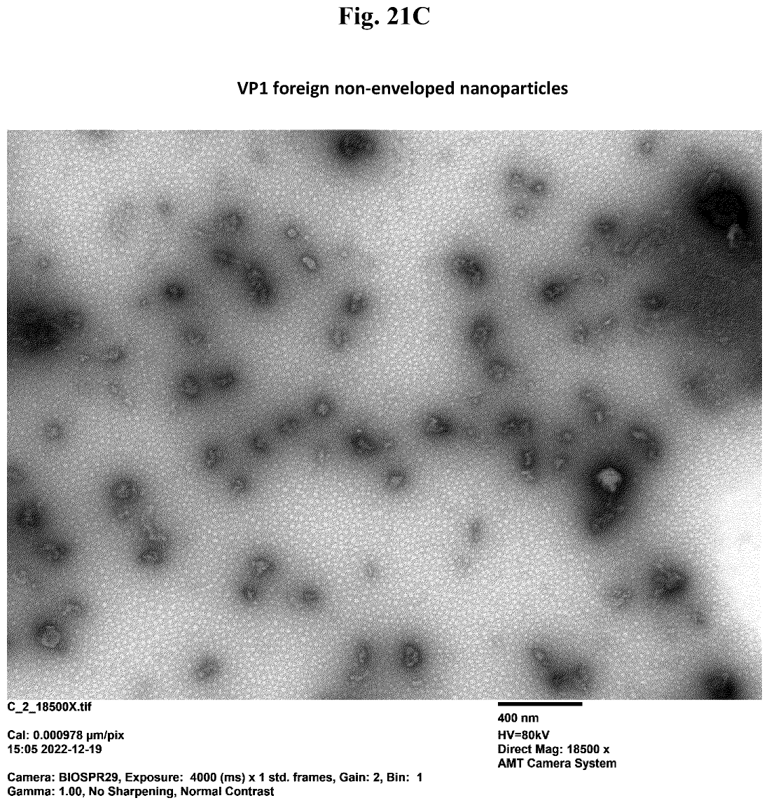

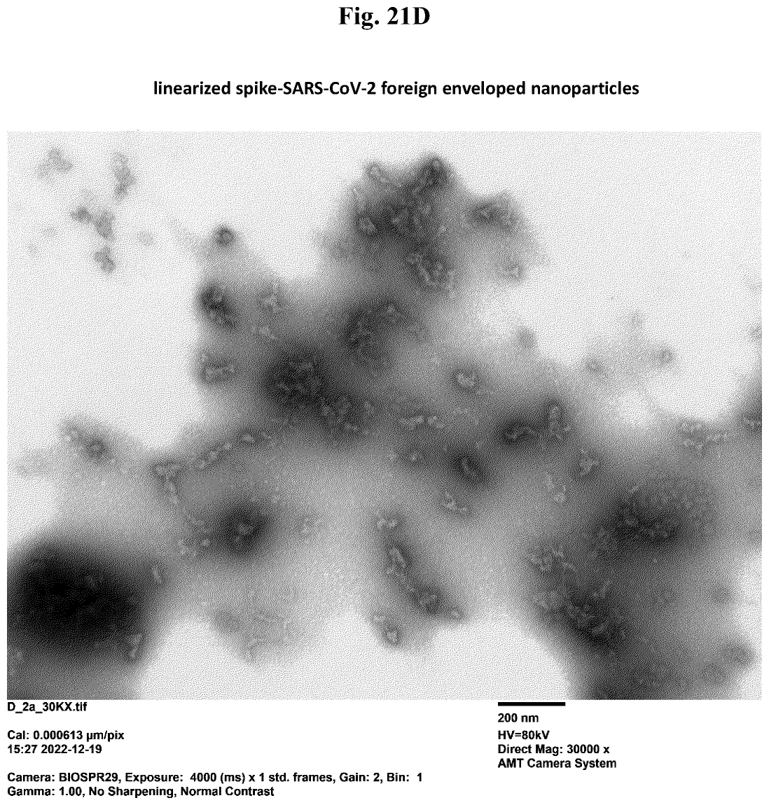

Another type of vesicle foreign nanoparticles was produced which rely on the assembly of proteins only. These proteins have the intrinsic ability to self-assemble in cells and/or in vitro in the absence of cells, to form a capsid (FIG. 3C). The interior of the structure may contain other proteins such as fluorescent proteins or luminescent enzymes. These proteins which compose these complex capsid-type biological structures possess a three-dimensional conformation and native post-translational modifications. This type of complex biological structure is used to simulate the surface of non-enveloped viruses, in the neutralizing molecules detection assay. Norovirus (a non-enveloped virus) foreign nanoparticles were produced, which express VP1 on their surfaces (discussed further in Example 9). A transmission electron microscopy image of these nanoparticles is shown in FIG. 21C.

These nanoparticles were made by engineering proteins and culture of eukaryotic or prokaryotic cells. Briefly, eukaryotic, or prokaryotic cells were modified to overexpress native, exogenous and/or chimeric proteins. To manufacture the complex biological structures of membrane type, specific overexpressed proteins were assembled onto the inner surface and/or through the membrane envelope. In addition, some of the overexpressed proteins play a part in the membrane budding process, resulting in the formation, and shedding of those vesicular shaped nanoparticles, in the extracellular media. These vesicular forms were linearized due to mechanical forces induced during their production and extraction. A linear form of the SARS-CoV-2 foreign nanoparticle expressing the Spike protein was produced and transmission electron microscopy image of these nanoparticles is shown in FIG. 21D.

To manufacture the complex biological structures of capsid type, proteins auto assembled inside the producer cells to form those structures, prior to being expulsed in the extracellular media. Along with the plasma membrane from the host producer cells, host nanoparticles were engineered to express surface receptors and proteins which aided in the recognition of surface ligands/proteins on the surface of foreign nanoparticles. These nanoparticles did not possess any genetic material (DNA or RNA) or any machinery allowing for functional replication. For example, for the SARS-CoV-2 model, host nanoparticles were engineered to express angiotensin converting enzyme 2 (ACE2) receptor and transmembrane serine protease 2 (TMPRSS2) for recognition to the SARS-CoV-2 Spike glycoprotein (mutant D614G) expressed on the surface of foreign nanoparticles. Since the nanoparticles were derived directly from the producer cells expressing these proteins, the surface proteins possessed all of the necessary post-translational modifications. In another model, foreign nanoparticles were engineered to express the vesicular stomatitis virus (VSV)-G surface glycoprotein and host nanoparticles were engineered to express VSV-G surface receptors. To produce a fluorescent signal in the host or foreign nanoparticles, GFP was fused to Gag or the surface receptor/foreign ligand expressed. For example, for the SARS-CoV-2 model, a plasmid for the expression of Gag fused to the fluorescent protein GFP and a plasmid for the expression of the SARS-CoV-2 Spike variant D614G were transfected into the producer cells. Other known fluorescent proteins, such as luciferase or secretory alkaline phosphatase (SEAP), were used to produce a fluorescent signal.

### Example 2: Characterization of Foreign (F) Nanoparticles

To demonstrate the ability of foreign nanoparticles to interact with natural/living cells, (i.e., binding to the plasma membrane), increasing amounts of fluorescent VSV viral nanoparticles, produced as described in Example 1, were incubated with HEK293T cells (10 000 cells per well in 96-well plate). Fluorescent viral nanoparticles lacking any specific antigen or ligand were used as a negative control to evaluate any non-specific interactions. HEK293T cells are naturally sensitive to the VSV infection since they possess receptors recognizing VSV glycoproteins. After incubation (18 h at 37° C., 5% CO₂) of the cells with the VSV nanoparticles, the culture was washed to remove any non-specific binding and subsequently analyzed by flow cytometry. The incubation was performed by the following protocol:
Day 1: Seed HEK293T cells at 10 000 cells per well in 96-well plate, incubation 24h at 37° C., 5% CO₂
Day 2: Dilute fluorescent VSV nanoparticles (antigenic/ligand and negative model) in complete medium
   Remove medium from the 96-well plate
   Drop diluted fluorescent VSV nanoparticles on HEK293T cells
   Incubate 18 h at 37° C., 5% CO₂
Day 3: Remove medium from the 96-well plate
   Wash adherent HEK293T cells with PBS containing 0.01% SDS and 10 mM EDTA to remove non-specific binding, then wash with only PBS and finally add complete medium
   Capture image by fluorescent microscopy
   Wash adherent HEK293T cells with PBS
   Add trypsin and incubate at 37° C. to detach cells
   Inhibit trypsin with complete medium
   Harvest cells by centrifugation (remove supernatant), and resuspend cells in PBS
   Analyze cells by flow cytometry for fluorescent signal (GFP)

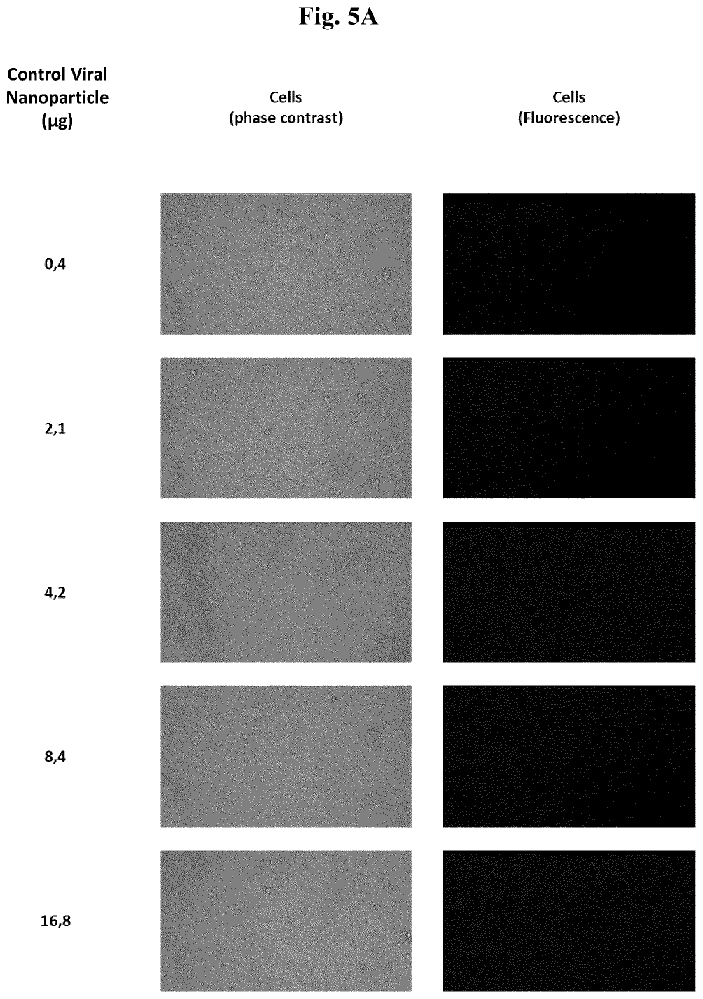

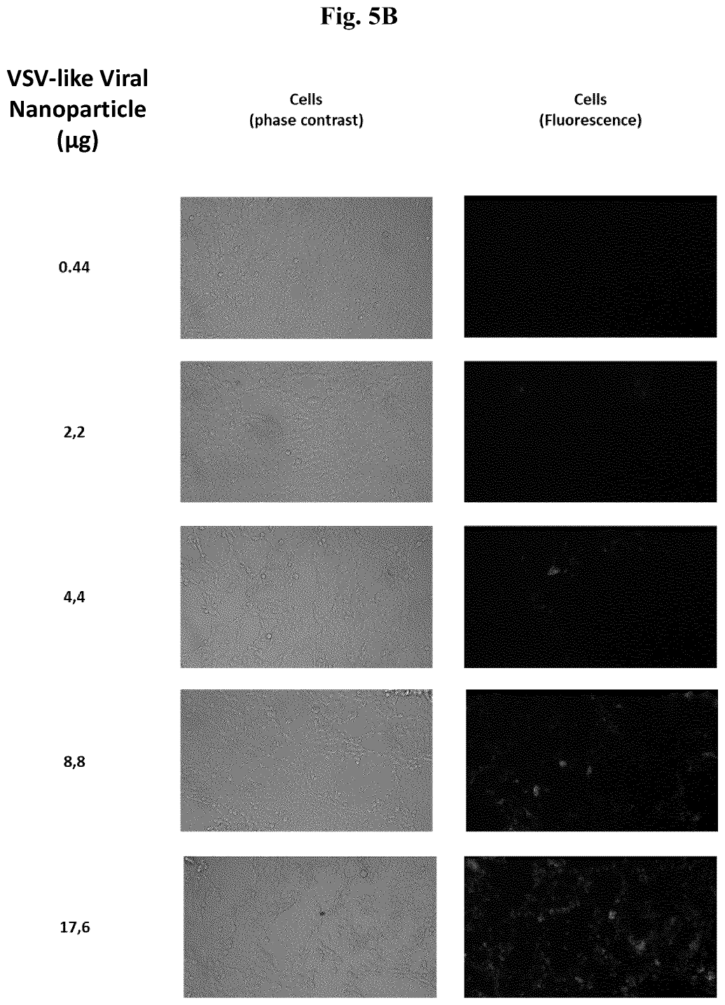

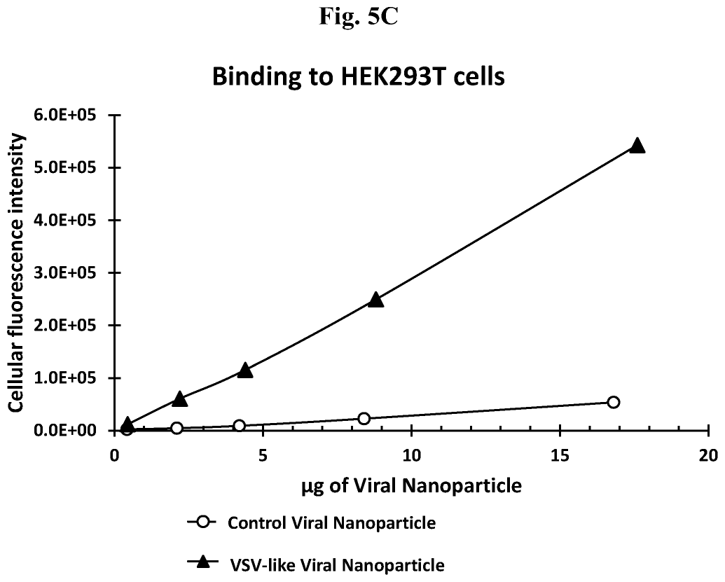

Cells incubated with fluorescent viral nanoparticles lacking the VSV G glycoprotein show a low fluorescent signal under microscopic observation (FIG. 5A), thereby indicating the absence of binding. Cells incubated with the fluorescent VSV nanoparticles expressing the VSV G glycoprotein showed an increasing fluorescent signal with the increasing quantity of nanoparticles (dose-response effect) (FIG. 5B). In addition, images revealed that the fluorescent signal was specifically located on the cells (as seen by phase contrast), either by surface binding or internalization, and not on the plastic supports. Although, flow cytometry analysis does not identify the cellular location of fluorescent viral nanoparticles (membrane and/or inside cells), these results indicated that the VSV nanoparticles were able to specifically interact with living cells (FIG. 5C).

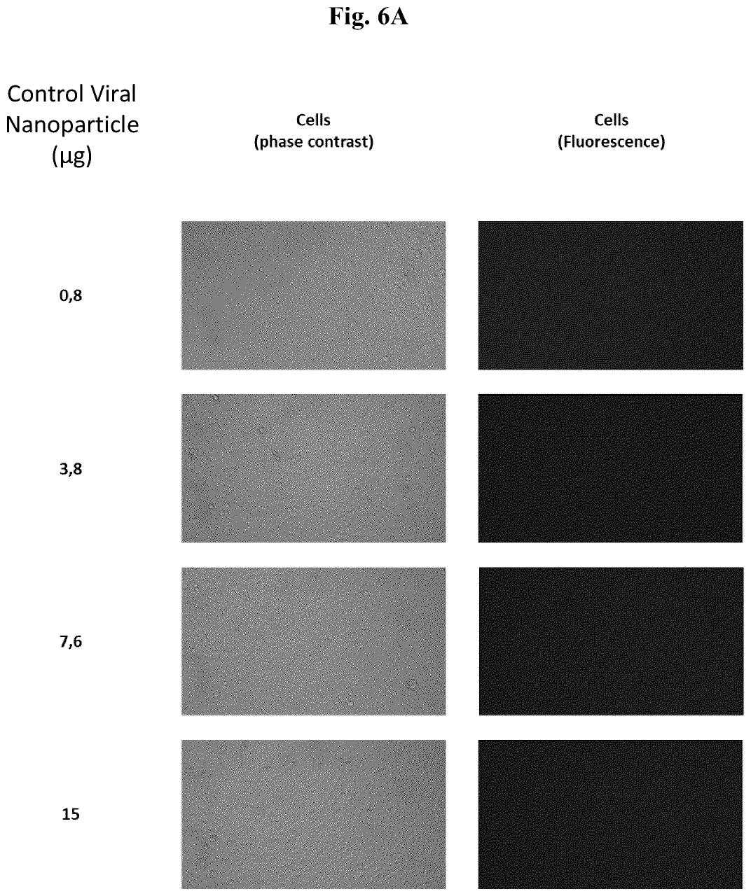

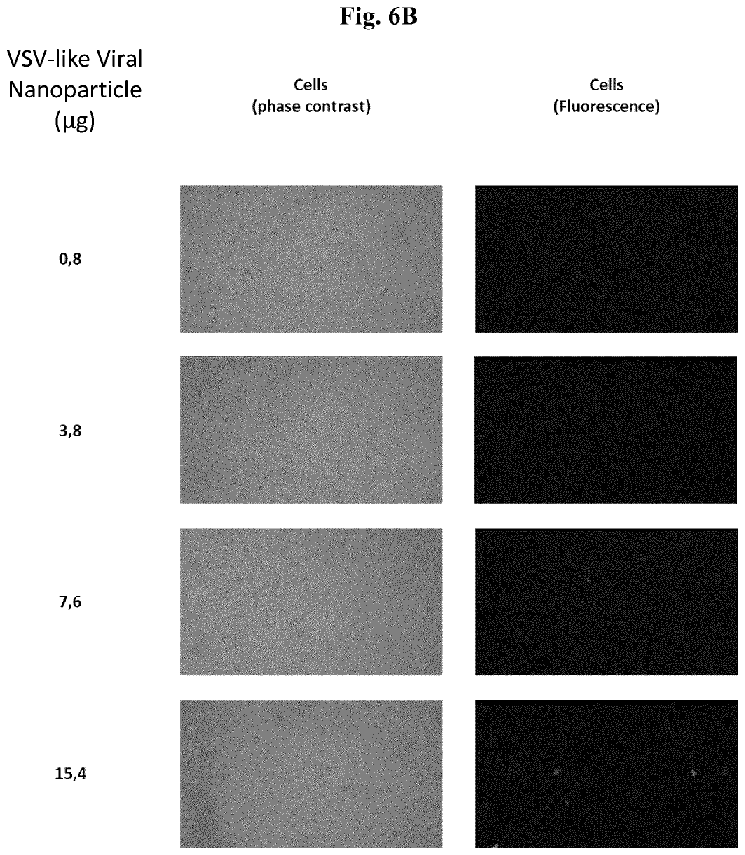

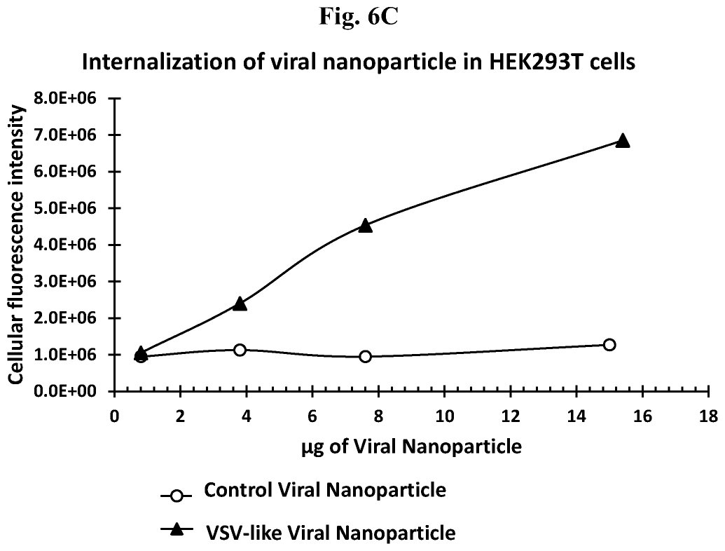

To determine whether viral nanoparticles were able to penetrate living cells or become internalized, fluorescent VSV nanoparticles expressing a plasmid containing a trans-activable GFP-expression cassette were incubated with HEK293T cells as described above. Since HEK293T cells possess the machinery for translation of the functional GFP protein, successful internalization of viral nanoparticles would be indicated by GFP expression. As seen in FIGS. 6B and 6C, GFP expression was observed by fluorescent microscopy and flow cytometry upon incubation of HEK293T cells with VSV nanoparticles expressing VSV G glycoprotein, but not with the negative control viral nanoparticles lacking VSV G glycoprotein (FIG. 6A). These data confirm that viral nanoparticles may be internalized upon surface recognition and that intracellular contents of viral nanoparticles may be shared with the host cell.

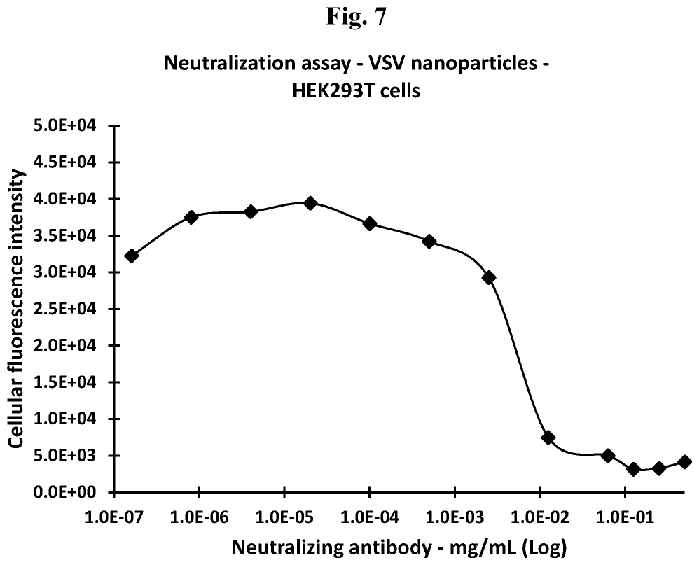

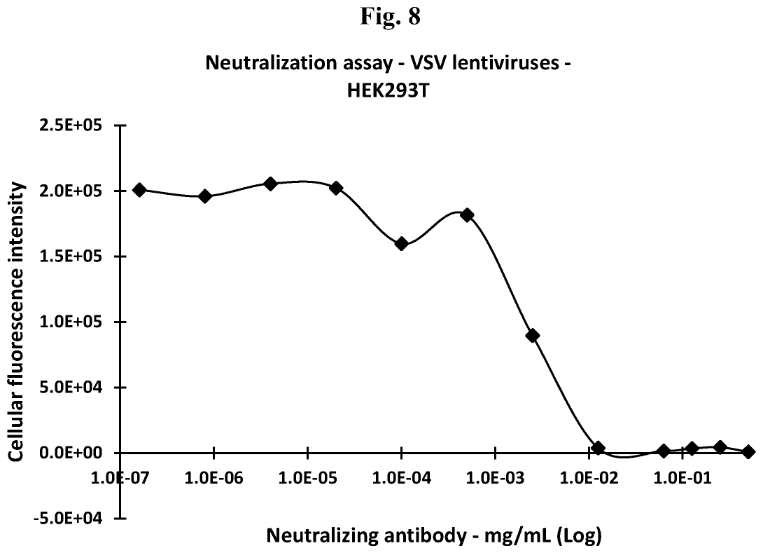

Finally, to determine whether binding of viral nanoparticles to cells can be neutralized, fluorescent VSV nanoparticles expressing the VSV G glycoprotein were preincubated with different concentrations of anti-VSV neutralizing antibody specific for VSV G-protein (Ab01402-2.0; clone IE9F9) for 1 h prior to incubation with host HEK293T cells. As shown in FIG. 7, after washing of the cells, increasing concentrations of neutralizing antibody inhibited binding of VSV nanoparticles to HEK293T cells, as demonstrated by the decrease in fluorescence by flow cytometry. To confirm these results, VSV-G-pseudotyped lentiviruses encapsulating a trans-activatable GFP-expressing cassette were produced and incubated with anti-VSV neutralizing antibody prior to incubation with HEK293T cells. This data demonstrates that the assay can be used to detect binding agents that prevent internalization of the foreign nanoparticles. Similar results were observed whereby a decrease in fluorescence was seen with increasing concentrations of neutralizing antibody (FIG. 8).

### Example 3: Detection of Neutralization of Host and Foreign Nanoparticle Interaction

First, binding of foreign nanoparticles to host nanoparticles was assessed by incubating fluorescent VSV nanoparticles with host nanoparticles produced as described in Example 1. Host nanoparticles were first coated onto the bottom of 96-well plates prior to incubation with VSV nanoparticles. The culture was done using the following protocol:
Day 1: Coat host nanoparticles in 96-well plate with the coating buffer overnight at 4° C.
Day 2: Remove liquid from the 96-well plate
   Add different concentrations of the fluorescent VSV nanoparticles into the wells Incubate overnight at 4° C.
   Remove liquid from the 96-well plate
Wash with PBS
Read fluorescence with a plate reader instrument

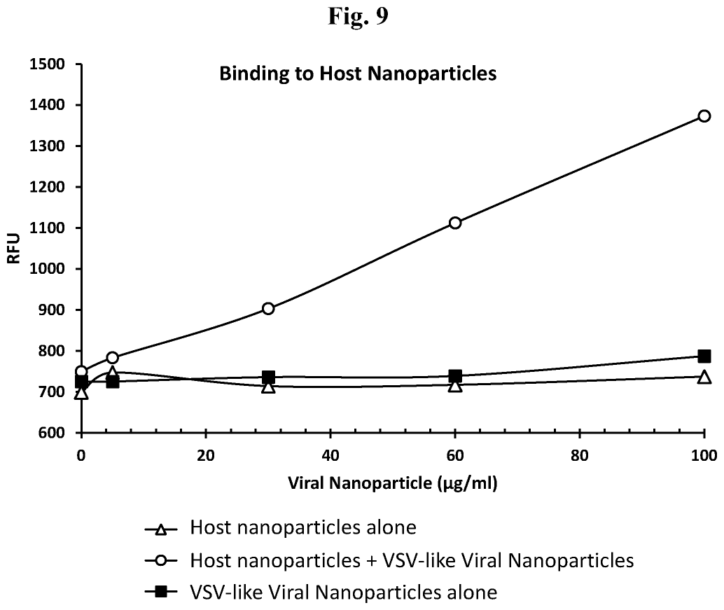

As shown in FIG. 9, increased fluorescence was observed with increasing amounts of VSV nanoparticles incubated with host nanoparticles, indicating binding of the two nanoparticles.

Next, to determine whether neutralization can be detected, the SARS-CoV-2 model of nanoparticles was used, as described in Example 1. Briefly, host nanoparticles exposing on their surface the ACE2 receptors and TMPRSS2 that bind the Spike glycoprotein of the SARS-CoV-2, have been coated in 96-well plate. Then, a defined quantity of the SARS-CoV-2 fluorescent nanoparticles was mixed and incubated with different concentrations of different biological samples containing or lacking neutralizing antibodies. After incubation, wells were washed to remove non-specific binding and analyzed for fluorescence detection with a plate reader instrument. If the inhibition of binding between the viral and host nanoparticles occurred (i.e., via neutralization antibodies), then the absence or decrease in fluorescence would be observed. The assay was conducted using the following protocol:
Day 1: Coat host nanoparticles in 96-well plate with the coating buffer overnight at 4° C.
Day 2: Incubate the fluorescent SARS-CoV-2 viral nanoparticles with increasing concentration of biological samples for 4 h at 37° C.
Remove liquid from the 96-well plate
   Wash with PBS
Add the preincubated mixes of viral nanoparticles/neutralizing antibody to the host nanoparticles
Incubate 4 h at 37° C.
Wash with PBS
Read fluorescence with a plate reader instrument

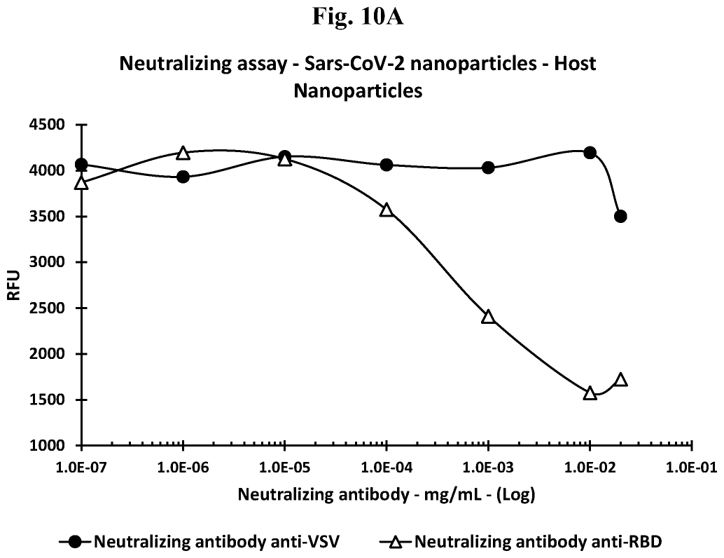

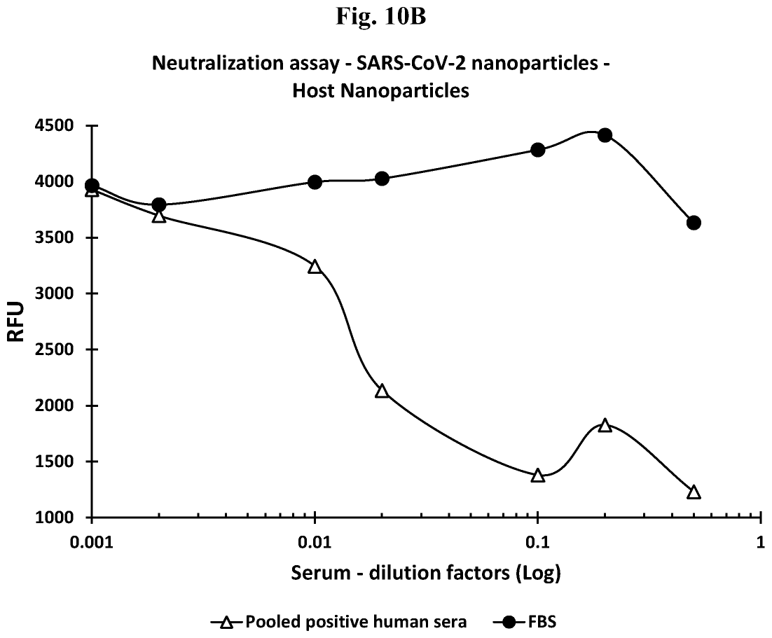

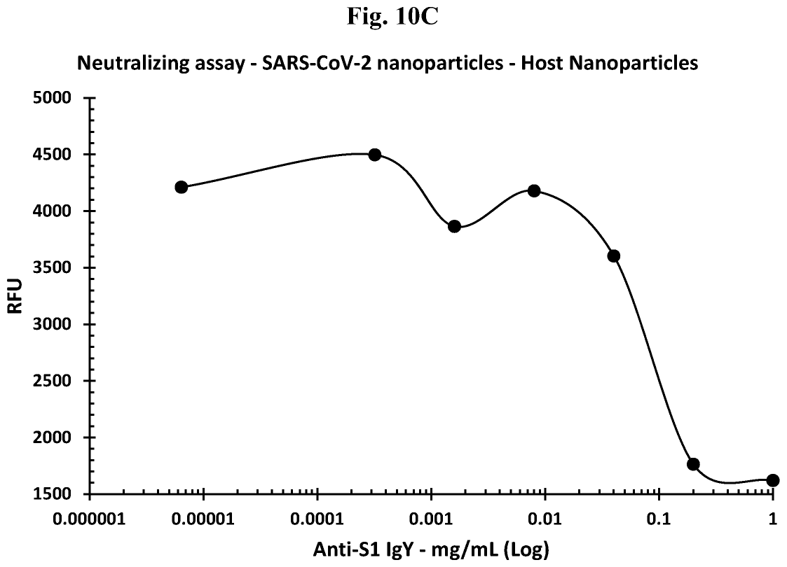

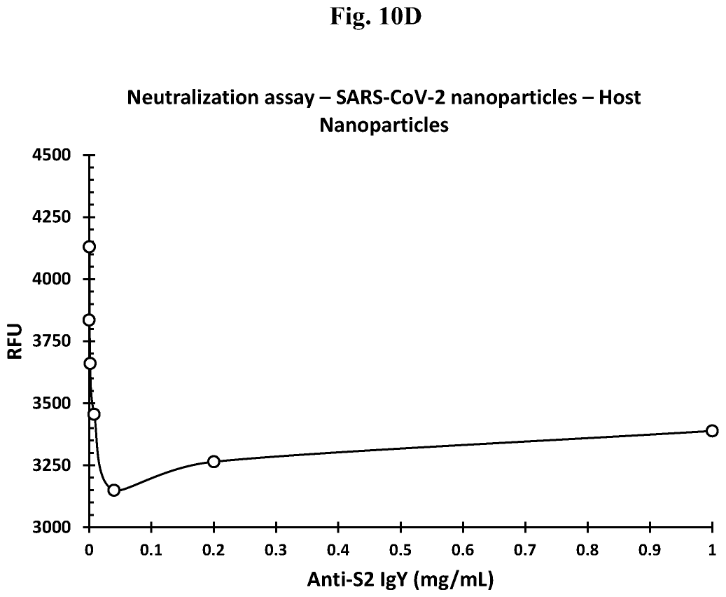

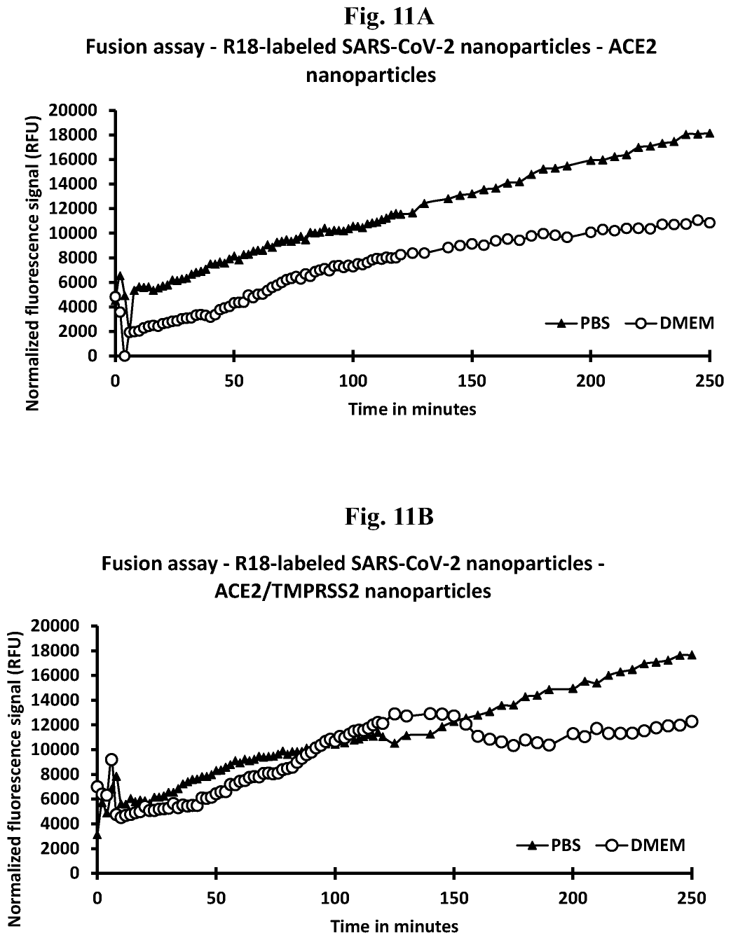

Neutralization of host and viral nanoparticle binding was observed with the addition of increasing concentrations of anti-Spike RBD antibody (human IgG monoclonal; Ab02019-12.1) (FIG. 10A), as shown by the decreasing fluorescent levels. Neutralization, however, was not observed in the presence of other antibodies, such as the anti-VSV IgG antibody (Ab01402-2.0). These data demonstrate that neutralization via antibodies can be detected with this assay.

To determine whether neutralizing agents can be detected within a complex biological sample, such as serum, the same experiment was conducted by incubating fluorescent SARS-CoV-2 nanoparticles with either pooled human sera from SARS-CoV-2 positive patients or with FBS. These samples were pre-tested for the presence of anti-SARS-CoV-2 antibodies. As shown in FIG. 10B, a decrease in fluorescence was observed with increasing concentrations of pooled human sera, as compared to FBS.

Finally, to determine whether this assay was useful in detecting neutralization via antibodies of different isotypes, species of origin, or target on Spike protein, the same experiment was conducted using purified chicken polyclonal IgY antibodies target the S1 or S2 region of the Spike protein of SARS-CoV-2. As shown in FIGS. 10C and 10D, neutralization via anti-S1 or anti-S2 was readily detected.

These data demonstrate that this assay can be safely and efficiently used to detect a variety of neutralizing agents present in complex biological assays.

### Example 4: Detection of Neutralization of Host and Foreign Nanoparticle Interaction

AS used herein, the term “enveloped” in the expression “foreign enveloped nanoparticles” describes nanoparticles aiming at mimicking enveloped viruses. Foreign enveloped nanoparticles produced were composed of a lipid membrane with biological molecules coming from the lipid membrane of the producer cells. In these experiments, the foreign enveloped nanoparticles display at their surface the SARS-CoV-2 spike glycoproteins anchored to the lipid membrane and have been produced using the ectopic co-expression of the Gag proteins and viral glycoproteins in mammalian cells. However, instead of GFP, SARS-CoV-2 foreign nanoparticles were modified with a fluorescent dye, octadecyl rhodamine B chloride (R18). This fluorescent dye was inserted into the foreign nanoparticles' membrane at a surface density that causes self-quenching of the fluorescent dye. Upon fusion between the labeled foreign nanoparticle's membranes and non labeled target membranes (host nanoparticles), relief of self quenching will occur, resulting in a proportional increase in the relative fluorescence intensity. Hence, the assay allowed us to confirm that the fusion process between foreign and host nanoparticles occurs in a direct manner. Furthermore, a transmission electron microscopy image of enveloped foreign SARS-CoV-2 nanoparticles expressing the Spike protein is shown in FIG. 21A.

Host ACE2 and ACE2/TMPRSS2 nanoparticles were produced as described in Example 1. A transmission electron microscopy image of enveloped host ACE2 nanoparticles is shown in FIG. 21B.

The fusion assays consisted of incubating host nanoparticles with foreign nanoparticles at 37° C. for 3 hours in suspension (i.e., no coating step) in either PBS or medium (DMEM). The mixes were performed in microwell plates. The luminescent signal was detected over time after the substrate addition, using a plate reader instrument (emission=400 nm). As shown in FIG. 11A, fusion between foreign Spike SARS-CoV-2 nanoparticles and host ACE2 nanoparticles was confirmed, both in PBS and DMEM. Results shown represent normalized fluorescence levels from controls (background fluorescence signal from nanoparticles in monocultures). Fusion was also successfully confirmed between foreign Spike SARS-CoV-2 nanoparticles and host ACE2/TMPRSS2 nanoparticles, in both PBS and DMEM (FIG. 11B).

The fusion between the host and foreign enveloped nanoparticles was also demonstrated using a split luciferase complementation assay based on the NanoLuc™ luciferase (Sasaki, M et al., 2018). This method is a well-characterized approach to study protein-protein interactions. The NanoLuc luciferase (Nluc) is a relatively small protein (around 19 kDa) that can produce bright luminescence. Nluc can be split into two non functional subunits, a large (18 kDa) subunit termed LgBiT and a small (11-amino acid) one termed HiBiT, creating a complementation reporter system for studying protein-protein interactions. This Nluc split complementation system was employed to successfully monitor the cellular entry and release of virus-like particles and viral particles.

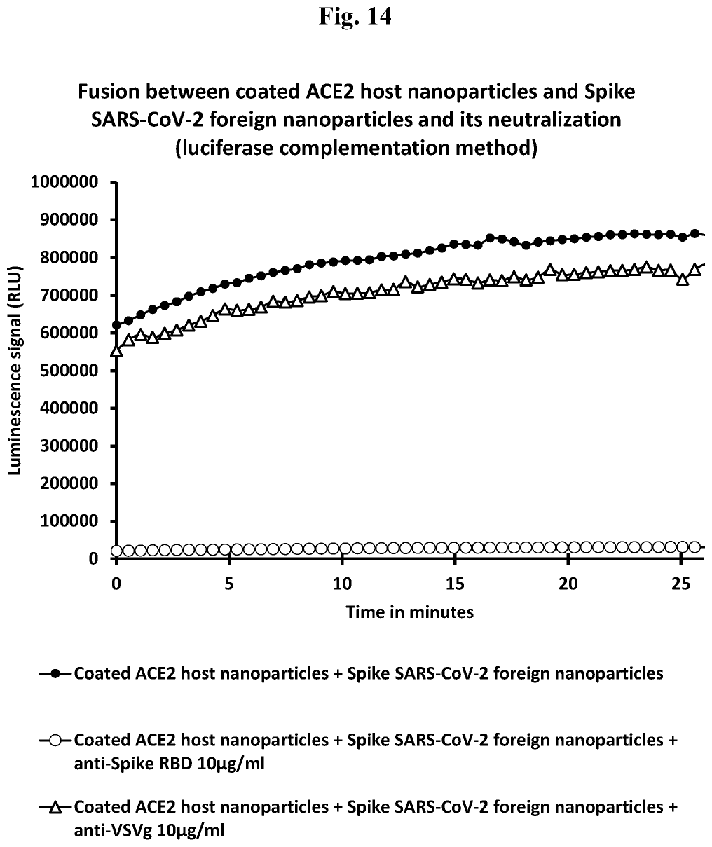

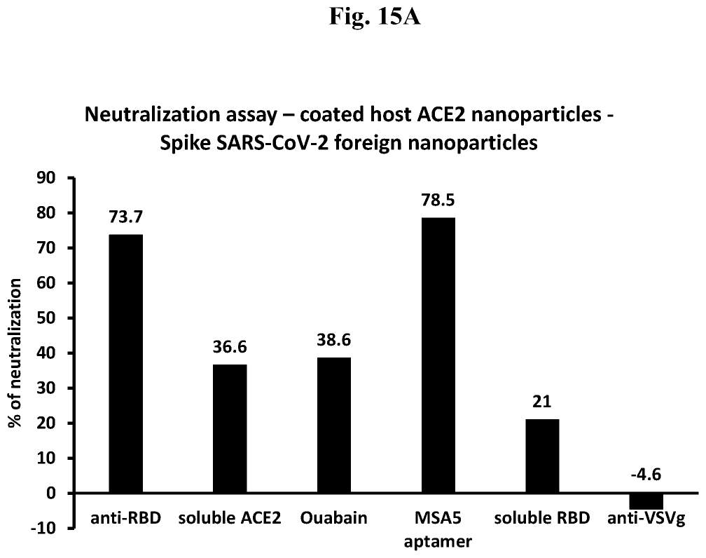

In the following experiments, the LgBiT and HiBiT fragments have been produced in fusion with the Gag protein during the nanoparticle's productions. Hence, the ACE2 host enveloped nanoparticles encapsulate the LgBiT protein fragment and the Spike SARS-CoV-2 foreign enveloped nanoparticles encapsulate the HiBiT protein fragment. If fusion occurs between the host and the foreign nanoparticles, following interaction between ACE2 receptors and Spike SARS-CoV-2 antigens, the LgBiT and HiBiT fragment could come together to form an active enzyme and generate a bright luminescent signal in the presence of substrate. To ensure a specific detection of fused nanoparticles, a non-lytic detection reagent (Nano-Glo® Live Cell Assay System—Promega) able to penetrate across lipid membrane without any disruption was used to measure luminescence production over the time. As shown in FIG. 14, Fusion between foreign Spike SARS-CoV-2 nanoparticles and coated host ACE2 nanoparticles was confirmed by this method.

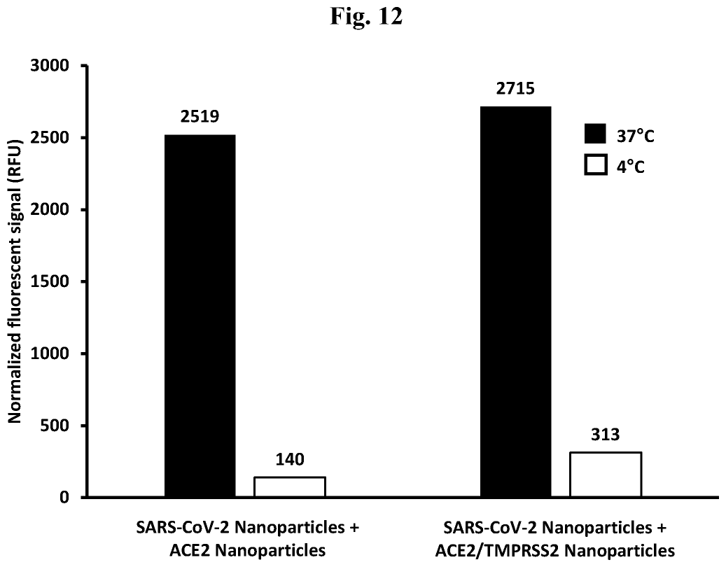

Fusion between enveloped host and foreign nanoparticles was further confirmed in FIG. 12. Fusion of native viruses and living cells is known to be temperature dependent since low temperatures constrain the mobility of viral glycoproteins. Moreover, it is well described that low temperatures inhibit any active biological pathway (energy-dependent) such as the cell infection by enveloped viruses.

To determine if the fusion between the foreign enveloped and the host nanoparticles involve protein motility and other active biological pathways such as it occurs for the natural infection process, fusion assays at 4° C. and 37° C. were conducted. Briefly, R18-labeled foreign enveloped nanoparticles were mixed with host nanoparticles and incubated at 37° C. or 4° C. for 3 hours. After the incubation time, formaldehyde was added to stop any biological process. Then, the fluorescent signal was detected using a plate reader (excitation=544 nm, emission=590 nm). As shown in FIG. 12, fusion was shown to occur at 37° C. with both ACE2 and ACE2/TMPRSS host nanoparticles, but was drastically diminished at 4° C.

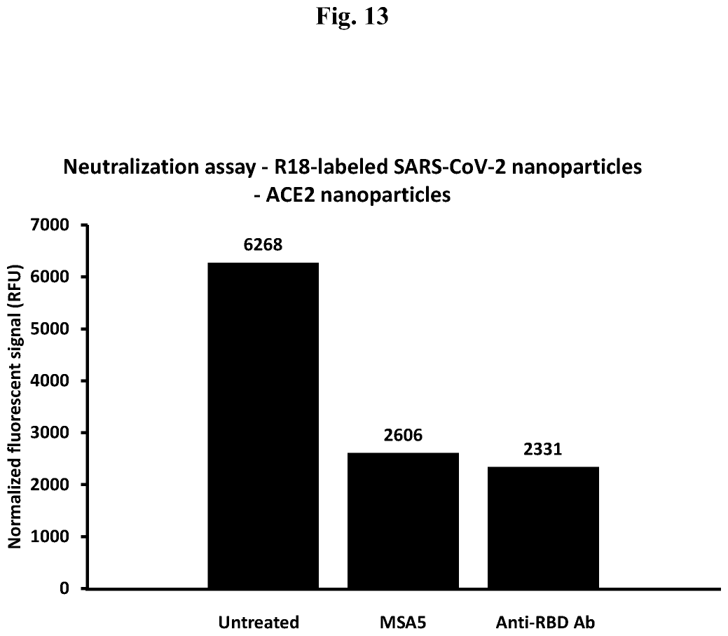

Next, neutralization of the fusion between foreign and host nanoparticles induced by both methods was conducted. As shown in FIG. 13, fusion between Spike SARS-CoV-2 foreign nanoparticles and coated host ACE2 nanoparticles in suspension was neutralized in the presence of anti-Spike RBD antibody (Seydoux et al., 2020) or a SARS-CoV-2 Spike protein specific-aptamer (MSA5; (Li et al., 2021)), compared to untreated controls. As shown in FIG. 14, fusion between coated ACE2 host nanoparticles treated with SARS-CoV-2 foreign nanoparticles (via the luciferase complementation system) in the presence of anti-Spike RBD antibody, but not an anti-VSVg antibody, was significantly inhibited.

These data suggest that foreign and host nanoparticles exhibit fusion processes similar to natural viral infection, either in suspension or when coated on a surface. Furthermore, this system may be successfully used to detect neutralizing agents.

### Example 5: Detection of Different Types of Neutralizing and Blocking Agents

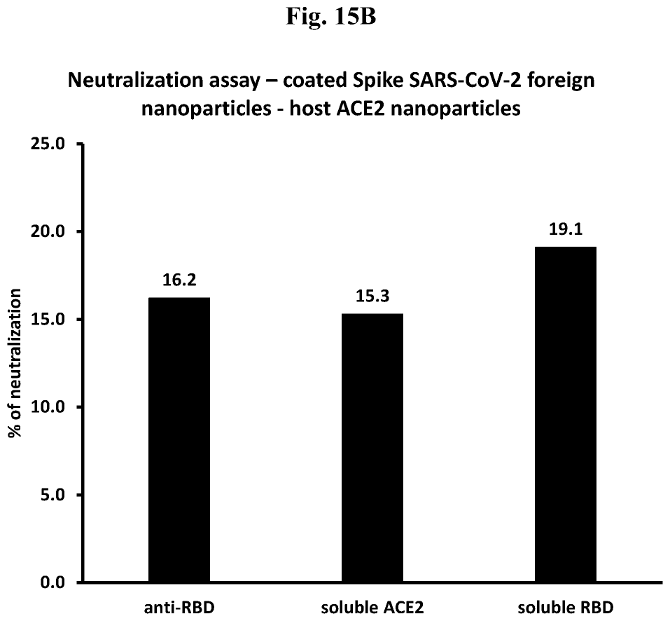

The system described herein can be used to detect neutralizing agents, like antibodies, but can also be used to detect different types of neutralizing agents. As shown in FIG. 15A, neutralization of Spike SARS-CoV-2 foreign nanoparticles and coated host ACE2 nanoparticles fusion was demonstrated using anti-Spike RBD antibody, soluble ACE2 protein (Chaouat et al., 2021), or a SARS-CoV-2 Spike protein specific-aptamer (MSA5; (Li et al., 2021)), compared to an anti-VSVg negative control antibody. Neutralization was further blocked in the presence of a small molecule, ouabain, which was shown to bind to SARS-CoV-2 Spike protein (Cathay et al., 2021). Furthermore, fusion of Spike SARS-CoV-2 foreign nanoparticles and coated host ACE2 nanoparticles was successfully blocked via a blocking agent, soluble RBD, which binds to the ACE2 receptor on the host nanoparticles (FIG. 15A).

The neutralization was then performed in a system whereby foreign nanoparticles were coated and the host nanoparticles were added in solution. As shown in FIG. 15B, with the addition of neutralizing agents like anti-RBD or soluble ACE2, or the blocking agent, soluble RBD, fusion of coated Spike SARS-CoV-2 foreign nanoparticles and host ACE2 nanoparticles was successfully neutralized/blocked.

These data suggest that the system described herein can be used to detect different types of neutralizing or blocking agents, such as antibodies, proteins, nucleic acids, and small molecules. Furthermore, the system can be adapted in different ways such as by coating either the foreign or host nanoparticles, or by having both nanoparticles in suspension.

### Example 6: Compatibility with Different Types of Biological Samples

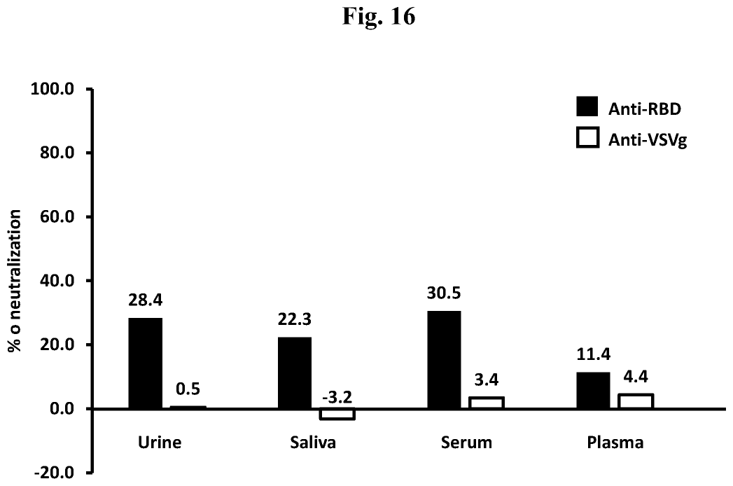

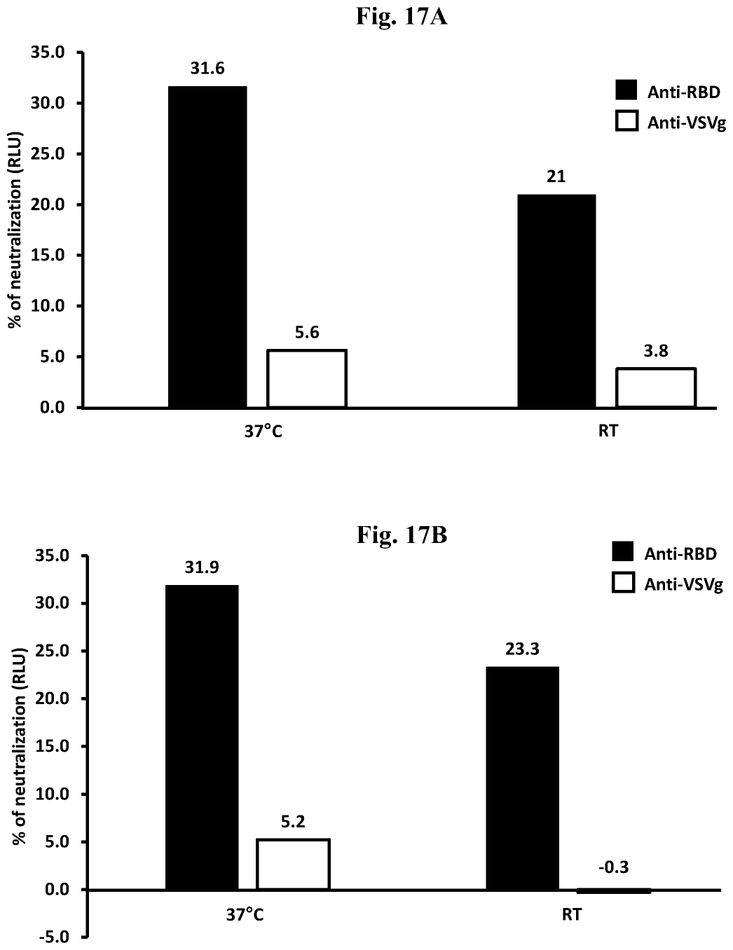

Neutralizing agents can be present in different bodily fluids. Therefore, the effectiveness of this system in different biological fluid samples was next determined. As shown in FIG. 16, neutralization of Spike SARS-CoV-2 foreign nanoparticles and coated host ACE2 nanoparticles was assessed in the presence of anti-Spike RBD Ab or anti-VSVg Ab (negative control) diluted in the either urine, saliva, or serum. Neutralization via anti-Spike RBD in all fluids was successfully shown. Similar results were observed in plasma, however, the effect was decreased likely due to the presence of heparin (which is not in serum), which was shown to inhibit SARS-CoV-2 entry into cells (Bewley et al., 2021). This effect was further apparent when the amount of plasma was increased (not shown).

These data suggest that the system may be used to detect neutralizing or blocking agents in different biological fluid samples.

### Example 7: Effect of Temperature and Incubation Timing

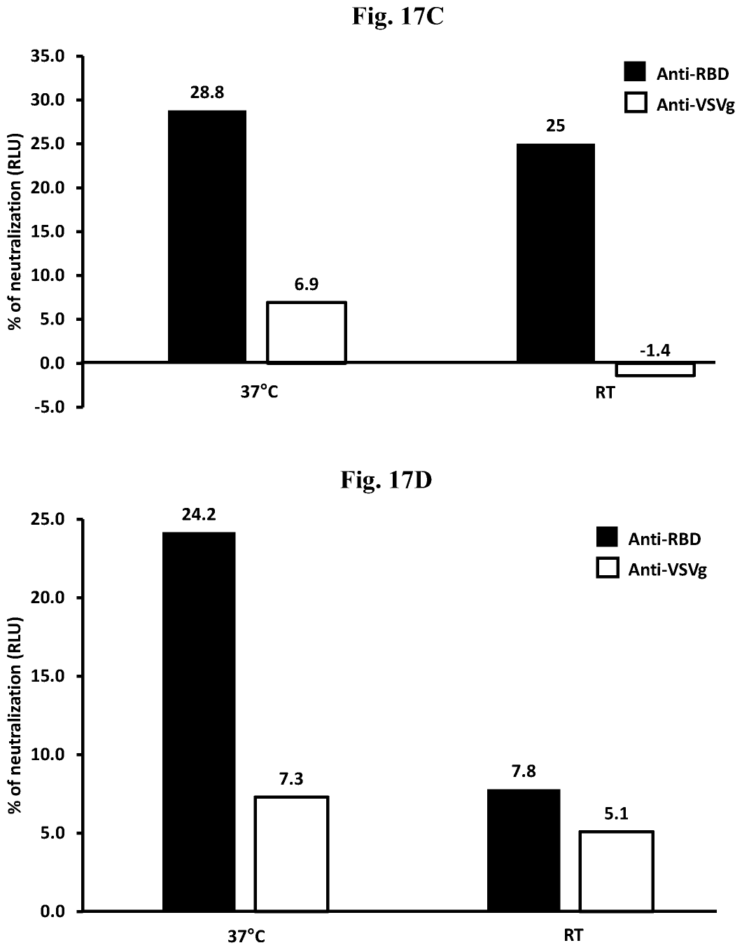

To further optimize the system, different temperature and incubation times were examined. As described in the previous Examples, foreign nanoparticles are generally incubated with the solution containing the neutralizing agent for 1 hour at 37° C. and the mix was then incubated with the host nanoparticles for 3 hours at 37° C. (FIG. 17A). Neutralization was also shown, with a slightly diminished effect, under these conditions at room temperature instead of 37° C.

Neutralization was maintained at both temperatures when the foreign nanoparticle and neutralizing agent incubation time was reduced to 30 minutes (FIG. 17B) or when the host nanoparticle and foreign nanoparticle/neutralizing agent incubation time was reduced to 1 hour (FIG. 17C). When both incubation times were reduced, neutralization effect was slightly reduced at 37° C., and significantly reduced at room temperature.

These data suggest that the system is very flexible in that it can be used at different incubation times of the different components, as well as either at 37° C. (e.g., in an incubator) or at room temperature (e.g., on the bench or in the field). Furthermore, incubation times may be increased or decreased to enhance the sensitivity of the system depending on the conditions (e.g., temperature).

### Example 8: Detection of Neutralizing Agents Against Non-Viral Foreign Nanoparticles

To determine whether this system can be adapted to detect neutralizing agents against microorganisms other than viruses, a bacterial foreign nanoparticle was developed.

Here, the system was used to detect neutralizing antibodies against the PA63 fragment (UniProt accession number: P13423) of the anthrax toxin from Bacillus anthracis. PA63 is a non-toxic fragment of the anthrax toxin that bind the host cell receptors (ANTXR2—UniProt accession number: P58335) and that is an antigen targeted by neutralizing antibodies.

In these experiments, the foreign nanoparticles were fluorescent enveloped nanoparticles decorated at their surface by PA63 fragments. Foreign enveloped nanoparticles were produced, similar to as described in Example 1, from HEK293 cells expressing the GAG proteins in fusion with the GFP and the chimeric proteins that enables the linkage between the targeted antigens (PA63 fragment) and the nanoparticle's surface. The chimeric proteins are composed of a membrane localization signal and a peptide tag able to form spontaneous amide bonds, based on harnessing reactions of adhesion proteins from the bacterium Streptococcus pyogenes. The resulting non-decorated nanoparticles are composed of a lipidic membrane from the producer cells that encapsulate the GFP (or another protein such as luciferase) and contain the chimeric proteins across the membrane, displaying the link tag at the outer surface. The targeted antigens are produced separately, and their sequence protein is in fusion with another peptide tag based coming from adhesion proteins of the bacterium Streptococcus pyogenes. The nanoparticles functionalization can be achieved following incubation with the targeted antigens. The two partner tags (on the nanoparticle surface and in fusion with the targeted antigens) form a spontaneous and irreversible isopeptide bond together. This functionalization method can be used independently of the antigen origin and can be applied to produce a range of nanoparticles decorated with protein originated from different sources such as bacteria, parasites, yeast, mammalian, or fungus.

The host nanoparticles were produced as previously described in Example 1, using the co-expression of GAG proteins and cell receptors (ANTXR2) targeted by PA63.

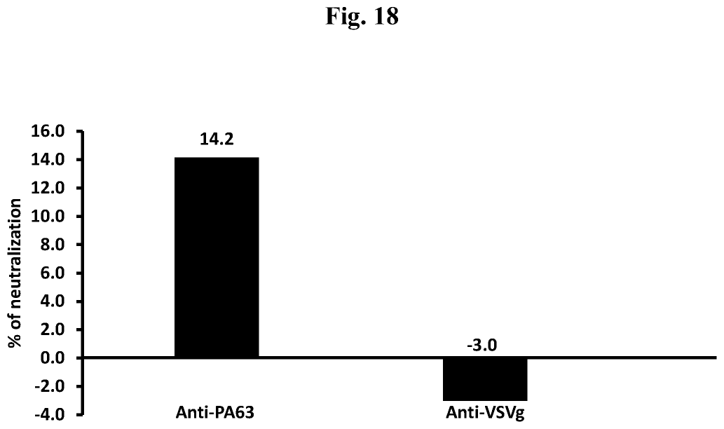

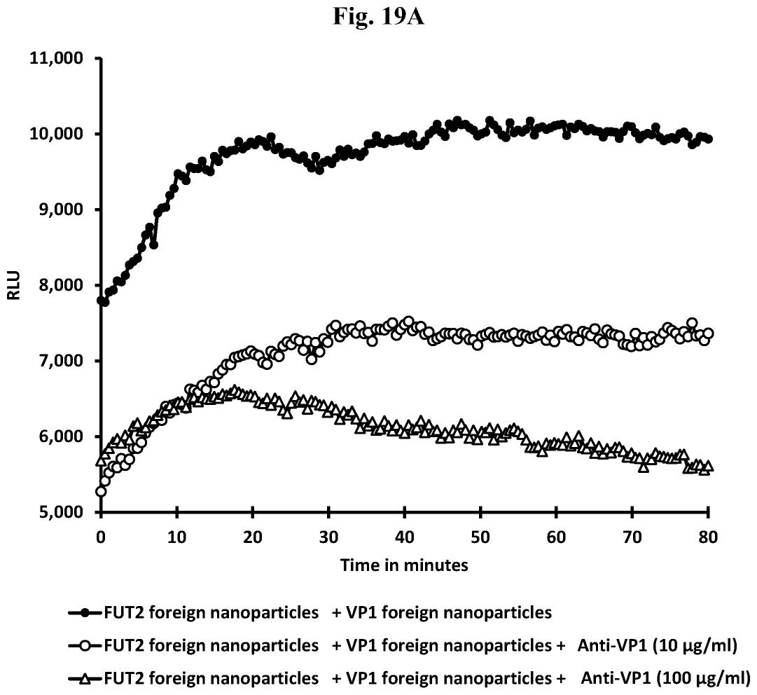

As shown in FIG. 18, neutralization of the interaction between coated host ANTXR2 nanoparticles and foreign B. anthracis PA63 nanoparticles was shown in the presence of anti-PA63 antibodies, but not with anti-VSVg antibodies (negative control).

These data suggest that this system may be used to detect neutralizing/blocking agents that neutralize/block the interaction of host cells with different microbial cells.

### Example 9: Detection of Neutralizing Agents Against Non-Enveloped Viral Foreign Nanoparticles

The structure of non-enveloped viruses consists of a protein capsid without any lipid membrane. To evaluate whether our system is effective in detecting neutralizing/blocking agents of non-enveloped viruses, foreign non-enveloped nanoparticles mimicking norovirus were used as well as host enveloped nanoparticles displaying the FUT2 receptors on their surface. The foreign non-enveloped nanoparticles were produced by expressing the norovirus capsid protein (VP1—UniProt accession number #Q83884) in HEK293 cells. Following its expression, VP1 can naturally auto-assembles without any other viral proteins to form the norovirus capsids. These capsids were used in this experiment as foreign non-enveloped nanoparticles.

The corresponding host nanoparticles were enveloped nanoparticles produced as described in Example 1, using the co-expression of Gag proteins and the cell receptors used by the norovirus (FUT2—UniProt Accession number #Q10981).

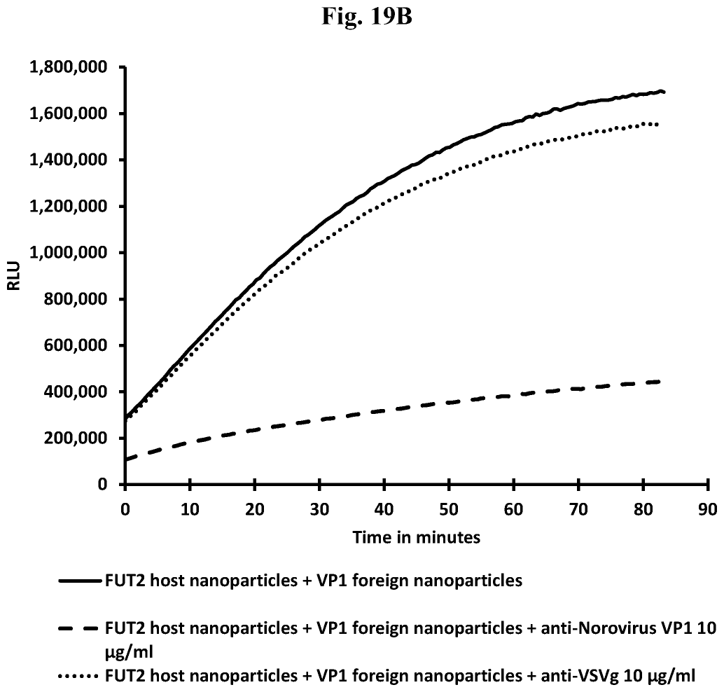

As shown in FIGS. 19A and 19B, neutralization of the interaction between coated host FUT2 nanoparticles and foreign norovirus VP1 nanoparticles was shown in the presence of anti-VP1 antibodies, but not with anti-VSVg antibodies (negative control).

These data suggest that this system may be used to detect neutralizing/blocking agents that neutralize/block the interaction of host cells with non-enveloped viruses.

### Example 10: Production of Host and Foreign Nanoparticles from Different Cell Types

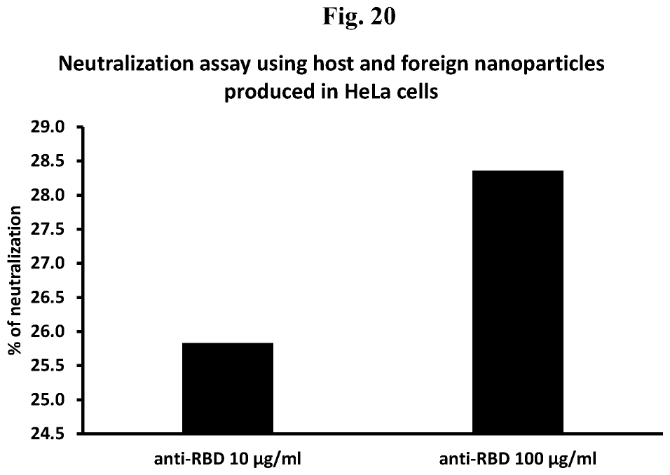

As shown in the previous Examples, host and foreign nanoparticles were produced from HEK293T cells. To determine whether other cells may be used to produce the nanoparticles, production of ACE2 host nanoparticles and SARS-CoV-2 Spike foreign nanoparticles was done using HeLa cells under the same conditions as Example 1. Neutralization of the interaction between the nanoparticles was then confirmed in the presence of anti-spike RBD antibodies (FIG. 20).

### REFERENCES

Bewley, K. R., Coombes, N. S., Gagnon, L., McInroy, L., Baker, N., Shaik, I., St-Jean, J. R., St-Amant, N., Buttigieg, K. R., Humphries, H. E., Godwin, K. J., Brunt, E., Allen, L., Leung, S., Brown, P. J., Penn, E. J., Thomas, K., Kulnis, G., Hallis, B., . . . Charlton, S. (2021). Quantification of SARS-CoV-2 neutralizing antibody by wild-type plaque reduction neutralization, microneutralization and pseudotyped virus neutralization assays. Nature Protocols, 16(6), Art. 6.
Caohuy, H., Eidelman, O., Chen, T., Liu, S., Yang, Q., Bera, A., Walton, N. I., Wang, T. T., & Pollard, H. B. (2021). Common cardiac medications potently inhibit ACE2 binding to the SARS-CoV-2 Spike, and block virus penetration and infectivity in human lung cells. Scientific Reports, 11(1), Art. 1.
Chaouat, A. E., Achdout, H., Kol, I., Berhani, O., Roi, G., Vitner, E. B., Melamed, S., Politi, B., Zahavy, E., Brizic, I., Rovis, T. L., Alfi, O., Wolf, D., Jonjic, S., Israely, T., & Mandelboim, O. (2021). SARS-CoV-2 receptor binding domain fusion protein efficiently neutralizes virus infection. PLOS Pathogens, 17(12), e1010175.
Li, J., Zhang, Z., Gu, J., Stacey, H. D., Ang, J. C., Capretta, A., Filipe, C. D. M., Mossman, K. L., Balion, C., Salena, B. J., Yamamura, D., Soleymani, L., Miller, M. S., Brennan, J. D., & Li, Y. (2021). Diverse high-affinity DNA aptamers for wild-type and B.1.1.7 SARS-CoV-2 spike proteins from a pre-structured DNA library. Nucleic Acids Research, 49(13), 7267-7279.
Sasaki, M., Anindita, P. D., Phongphaew, W., Carr, M., Kobayashi, S., Orba, Y., & Sawa, H. (2018). Development of a rapid and quantitative method for the analysis of viral entry and release using a NanoLuc luciferase complementation assay. Virus Research, 243, 69-74.
Seydoux, E., Homad, L. J., MacCamy, A. J., Parks, K. R., Hurlburt, N. K., Jennewein, M. F., Akins, N. R., Stuart, A. B., Wan, Y.-H., Feng, J., Nelson, R. E., Singh, S., Cohen, K. W., McElrath, M. J., Englund, J. A., Chu, H. Y., Pancera, M., McGuire, A. T., & Stamatatos, L. (2020). Characterization of neutralizing antibodies from a SARS-CoV-2 infected individual. BioRxiv: The Preprint Server for Biology, 2020.05.12.091298.

1. An in vitro system for the detection of binding agents (e.g., neutralizing or blocking agents that disrupt host-microbe interaction and/or fusion/penetration) in a fluid sample (e.g., biological fluid sample), said system comprising:
host nanoparticles comprising fragments of host cells expressing or engineered to express surface receptors recognized by a foreign ligand, wherein the host nanoparticles comprise plasma membrane fragments of the host cell expressing the surface receptors recognized by the foreign ligand; and
foreign nanoparticles expressing or engineered to express the foreign ligand on their surface such that the foreign ligands are recognizable by the surface receptors of the host nanoparticles;
wherein the host nanoparticles and/or the foreign nanoparticles further comprise a detectable label, and
wherein said binding agents are present in the sample when preincubation of either the host or foreign nanoparticles with the sample decreases binding/interaction between the host nanoparticles and the foreign nanoparticles as compared to a corresponding fluid sample lacking the binding agents.

2.-4. (canceled)

5. The system of claim 1, wherein the host nanoparticles are produced or derived from host producer cells.

6. The system of claim 5, wherein the host producer cells are eukaryotic (e.g., yeast or mammalian) cells.

7. The system of claim 5, wherein the host producer cells:
(a) engineered to express or overexpress the foreign ligand;
(b) engineered to express or overexpress the detectable label;
(c) engineered to express or overexpress an exogenous viral (e.g., from an enveloped or non-enveloped virus), bacterial, or fungal protein that induces extracellular vesicle or extracellular particle formation (e.g., biological nanoparticles or virus-like particles) in the host producer cell; or
(d) any combination of (a) to (c).

8. The system of claim 1, wherein the host nanoparticles and/or the foreign nanoparticles are vesicular nanoparticles comprising an exogenous viral structural protein (e.g., a late assembly (L) domain protein or polyprotein, such as HIV Gag) that induces host cell budding.

9. The system of claim 8, wherein the exogenous viral structural protein is fused to the detectable label.

10. The system of claim 1, wherein the foreign nanoparticles are produced or derived from a microorganism (e.g., bacteria, fungi, viruses, or parasites), from eukaryotic (e.g., yeast or mammalian) producer cells, or are formed by self-assembling viral structural proteins (e.g., capsid proteins).

11. The system of claim 10, wherein the microorganism or eukaryotic producer cells from which the foreign nanoparticles are produced or derived:
(a) engineered to express or overexpress the surface receptor;
(b) engineered to express or overexpress the detectable label;
(c) engineered to express or overexpress an exogenous viral (e.g., from an enveloped or non-enveloped virus), bacterial, or fungal protein that induces extracellular vesicle or extracellular particle formation in the host producer cell;
(d) are nanoparticles that fuse with and/or penetrate into the host nanoparticles upon contact therewith; or
(e) any combination of (a) to (d).

12. (canceled)

13. The system of claim 1, wherein the foreign nanoparticles are derived from SARS-CoV-2 virus and/or express or are engineered to express a SARS-CoV-2 Spike protein, or a portion thereof, on their surface, and/or wherein the host nanoparticles express angiotensin converting enzyme 2 (ACE2) and/or transmembrane serine protease 2 (TMPRSS2) on their surface.

14. (canceled)

15. The system of claim 1, wherein the foreign ligand is or comprises a surface ligand expressed on a microbial cell, an autoantigen, or an allergen.

16. The system of claim 1, wherein the detectable label is internally expressed or expressed on the surface of the foreign or host nanoparticles.

17. The system of claim 1, wherein the detectable label is or comprises a fluorescent or luminescent molecule (e.g., protein).

18. The system of claim 1, wherein the detectable label is detectable upon contact with a substrate, protein, or enzyme.

19. The system of claim 1, wherein the sample is or comprises blood, serum, tears, saliva, plasma, urine, nasal secretion, bronchoalveolar lavage fluid (BALF), fecal, or cerebral spinal fluid.

20. The system of claim 1, wherein the binding agent is or comprises an antibody or an antigen-binding fragment thereof, peptide, protein, nucleic acid, aptamer, ribozyme, or a small molecule.

21. The system of claim 1, wherein the binding agent:
(i) recognizes the surface receptor of the host nanoparticle or the foreign ligand of the foreign nanoparticle;
(ii) prevents or attenuates fusion of the membranes of the host nanoparticle and the foreign nanoparticle; or
(iii) prevents or attenuates internalization/penetration of the foreign nanoparticle into the host nanoparticle.

22.-24. (canceled)

25. The system of claim 1, wherein the host nanoparticles and/or the foreign nanoparticles are:
(i) immobilized or are for immobilization on a solid support; or
(ii) in suspension.

26.-27. (canceled)

28. The system of claim 8, wherein the exogenous viral structural protein that induces host cell budding is or comprises a late assembly (L) domain protein or polyprotein.

29. The system of claim 8, wherein the exogenous viral structural protein that induces host cell budding is or comprises HIV Gag.

30. A kit for the detection of binding agents in a fluid sample, said kit comprising:
a first container comprising the host nanoparticles as defined in claim 1; and
a second container comprising the foreign nanoparticles as defined in claim 1.
